(19) United States
(12) Patent Application Publication
(10) Pub. No.: US 2024/0277730 A1
(43) Pub. Date: Aug. 22, 2024

(54) SUPPRESSION OF COVID-19 REPLICATION BY COVID-19 ENTRY INHIBITORS

(71) Applicants: Board of Regents of the University of Nebraska, Lincoln, NE (US); THE CURATORS OF THE UNIVERSITY OF MISSOURI, Columbia, MO (US)

(72) Inventors: Kamlendra Singh, Columbia, MO (US); Siddappa Byrareddy, Omaha, NE (US); Arpan Acharya, Omaha, NE (US)

(21) Appl. No.: 18/548,657

(22) PCT Filed: Mar. 3, 2022
(86) PCT No.: PCT/US2022/018749

§ 371 Date: Sep. 1, 2023

Related U.S. Application Data
(60) Provisional application No. 63/200,366, filed on Mar. 3, 2021.

(51) Int. Cl. A61K 31/553; A61K 31/145; A61K 31/277; A61K 31/395; A61K 31/404; A61K 31/4174; A61K 31/428; A61K 31/4439; A61K 31/4709; A61K 45/06

(52) U.S. Cl. CPC A61K 31/553; A61K 31/145; A61K 31/277; A61K 31/404; A61K 31/4174; A61K 31/428; A61K 31/4439; A61K 31/4709; A61K 31/5545; A61K 45/06

(57) ABSTRACT

The present invention relates to compounds, compositions, and methods, for treating viral infections. In particular, entry inhibitor compounds are disclosed for treatment of coronavirus infections, including SARS-COV-1 and SARS-COV-2 infections. The compounds bind to the interface of a SARS-COV-2 spike protein receptor binding domain (RBD) and a host cell ACE-2 receptor. The entry inhibitor compounds show antiviral activity, favorable kinetics, and temporally act at the entry of SARS-COV-2 infection. In embodiments, the compounds are used as medicaments for the inhibition of viral replication including SARS-COV-1 and/or SARS-COV-2 replication, for the treatment or prophylaxis of viral infections including SARS-COV-1 and SARS-COV-2 infections, and/or for the treatment or prophylaxis of an illness due to SARS-COV-1 and SARS-COV-2 infections.

### CROSS REFERENCE TO RELATED APPLICATIONS

This is a U.S. National Phase application of PCT/US2022/018749, filed Mar. 3, 2022, which claims the benefit of priority of U.S. Provisional Patent Application Nos. 63/200,366, filed Mar. 3, 2021, which is incorporated herein by reference in its entirety.

### STATEMENT REGARDING FEDERALLY SPONSORED RESEARCH

This invention was made with Government support under AI113883, AI129745, and AI076199 awarded by the National Institutes of Health. The government has certain rights in the invention.

### REFERENCE TO SEQUENCE LISTING SUBMITTED ELECTRONICALLY

The instant application contains a Sequence Listing which has been submitted electronically in ASCII format and is herein incorporated by reference in its entirety. Said ASCII text copy, created on Apr. 4, 2024, is named “P13515US01_SequenceListing.txt” and is 924 bytes in size.

### FIELD OF THE INVENTION

The present invention relates to antiviral compounds for the treatment of SARS-COV infections, more specifically, to small molecule entry inhibitors of SARS-COV-2 that block the entry, replication and transmission of Wuhan-Hu-1 and variants of concern.

### BACKGROUND OF THE INVENTION

Severe acute respiratory syndrome coronavirus 2 (SARS-COV-2), the etiological agent of Coronavirus Disease 19 (COVID-19) emerged early December 2019, in Wuhan City, China. Since its emergence. SARS-COV-2 has claimed more than 2 million lives and caused unprecedented socio-economic loss globally. The Spike glycoprotein (S-protein), membrane protein (M), and envelope protein (E) make the spherical envelope of SARS-COV-2. The S-protein consists of S1 and S2 subunits. The S1 subunit contains two major domains: an N-terminal domain (NTD) and a receptor-binding domain (S-RBD) in addition to CTD1 (C-terminal domain 1) and CTD2 (C-terminal domain 2). In contrast, the S2 subunit contains fusion peptide (FP), heptad repeat 1 (HR1), central helix (CH), heptad repeat 2 (HR2), connector domain (CD), transmembrane domain (TM), and a cytoplasmic tail (CT). The S-RBD within the S1 subunit binds to the host cell receptor angiotensin converting enzyme 2 (ACE2) and facilitates viral entry into the host cell, while the S2 subunit mediates membrane fusion. Although the binding of S-RBD and ACE2 is a well-documented determinant of cellular entry, depending on cell types, the virions may enter the cell through clathrin-mediated endosomal pathways or clathrin-independent nonendosomal pathways.

SARS-COV-2 genome is a positive (+) sense single-stranded RNA (ssRNA) of ˜30 kilobases. It belongs to β-CoV lineage similar to the closely related SARS-COV that emerged in 2002-2003. Multiple open reading frames (ORFs) encode polyproteins (pp)1a and pp1ab. Polyproteins pp1a and pp1ab are processed by the viral proteases MPro and 3CL^{pro} into 16 non-structural proteins (nsps). Many of these nsps assemble to form a replication-transcription complex (RTC) within double membrane vesicles (DMVs) and generates a minus (−) sense RNA. The (−) sense RNA is subsequently used as a template for the synthesis of a (+) sense RNA genome and a set of segmented genomic RNA (sgRNA) with common 5′ leader sequence and 3′-end poly A sequence. The sgRNAs are translated into several structural proteins. Four major structural proteins: the membrane (M), nucleocapsid (N), envelope (E), and Spike protein (S protein) together with host cell membrane form an infectious mature virus particle.

Studies comparing the SARS-COV-2 genome MERS-COV and SARS-COV-1 have shown that SARS-COV-2 has a similar RBD structure to that of SARS-COV-1 (that emerged in 2002-2003), despite amino acid variations at some key residues. Genomic comparison of SARS-CoV-2 with SARS-COV-1 and bat SARS-like coronaviruses reveal that the S1 subunits of the spike proteins have a sequence identity of ˜75%. Owing to the function of S protein at entry and the maturation of the virus, almost all vaccination strategies target the S-protein RBD. Extraordinary measures initiated by the private sector and supported by government resources have resulted in the development of vaccine candidates with promising efficacy. However, the rapid and continued evolution of SARS-COV-2 continues to motivate the development of small-molecule entry inhibitors which are cost-effective, scalable, and less vulnerable to genetic drift.

### SUMMARY OF INVENTION

Disclosed herein are small-molecule entry inhibitor compounds which block viral replication in SARS-COV-2 and related variants. The compounds may be used to treat diseases and other conditions in a subject in need thereof, including subjects suffering from Coronavirus Disease 2019 (COVID-19).

More specifically, the invention pertains to certain small, low molecular weight compounds that bind to the interface of SARS Spike protein receptor binding domain (RBD) and host cell ACE-2 receptor. Utilizing a computer-aided drug design (CADD) approach, compounds were identified and validated as inhibitors of S-RBD and ACE-2 interaction.

Several compounds were selected for optimization based on docking scores and visual inspection of these compounds with S-RBD and ACE2. Specifically, formula IIa and IIIa scaffolds were used to develop small, low molecular weight compounds of the formulas shown in claims 5, 6, and 22. As disclosed below, these compounds include, but are not limited to: formula (IIb), formula (IIc), formula (IId), formula (IIe), formula (IIIb), and formula (IIIc). Additional disclosed compounds include but are not limited to: formula (Ia), formula (IV), formula (V), formula (VI), formula (VII), formula (VIII), formula (IX), and formula (X). The above compounds are adapted to inhibit S-RBD and ACE-2 interaction to varying degrees.

In some embodiments, three compounds (the formula (Ia), formula (IIa), and formula (IIb)) were shown to inhibit viral replication at a sub-micromolar IC₅₀. The other formula (IIa) derivatives (including formula (IIc), formula (IId), and formula (IIe)) may also block viral replication at a sub-micromolar IC₅₀. In other embodiments, formula (IIa) acts synergistically with remdesivir (RDV), providing an effective combination therapy.

Thus, in embodiments, some of the entry inhibitor compounds of the present invention will be useful for administration to a subject to treat or prevent infection with SARS-COV-2. Further, the compounds are effective against variants of SARS-COV-2, including the South African, Scotland, and Delta variants.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE FIGURES

In embodiments. FIG. 1A is a flow chart depicting an in-silico screening process used to identify SARS-COV-2 entry inhibitors. FIG. 1B shows a zoomed-out view of residues at the interface of S-RBD and ACE2. FIG. 1C shows a zoomed-in view of the pocket selected for compound docking. The dotted line shows the discontinuous backbone of the protein (S-RBD, cyan, and ACE2, green). FIGS. 1D-1I show molecular docking of five compounds selected based on their glide score for in vitro screening of antiviral activity against SARS-COV-2, including formula (Ia)(FIG. 1D), formula (IIa)(FIG. 1E), formula (V)(FIG. 1F), formula (VI)(FIG. 1G), formula (VI)(FIG. 1H), and formula (VII)(FIG. 1I). FIG. 1G shows a first binding orientation of formula (VI). FIG. 1H shows a second binding orientation of formula (VI). The yellow dotted lines represent the polar interactions formed by the compounds with the nearest protein residues. The ACE2 residues represented as sticks are green carbons, whereas S-RBD residues are colored as cyan sticks. The protein backbone is represented as ribbons (green=ACE2 and cyan=S-RBD). The entry inhibitor compounds are shown in ball-and-stick representations with magenta carbons. All other atoms are colored by atom type (red=oxygen, blue=nitrogen, orange=sulfur, turquoise=fluorine, and light green=chlorine).

FIGS. 2A-2P show a variety of SARS-COV entry inhibitor compounds and/or derivatives as disclosed herein. Provided are figures showing formula (Ia)(FIG. 2A), formula (IIa)(FIG. 2B), formula (IIb)(FIG. 2C), formula (IIc)(FIG. 2D), formula (IId)(FIG. 2E), formula (IIe)(FIG. 2F), formula (IIIa)(FIG. 2G), formula (IIIb)(FIG. 2H), formula (IIIc)(FIG. 2I), formula (IV)(FIG. 2J), formula (V)(FIG. 2K), formula (VI)(FIG. 2L), formula (VII)(FIG. 2M), formula (VIII)(FIG. 2N), formula (IX)(FIG. 2O), and formula (X)(FIG. 2P). See Table 2 for a summary of the above-disclosed compounds.

FIG. 3 depicts the screening of compounds including potential drug-like compounds selected via computer-aided drug design (CADD) for their ability to bind to the ACE2: SARS-CoV-2 Spike receptor-binding domain (RBD). In embodiments, formula (Ia), formula (IIa), formula (V), formula (VI), and formula (VII) are tested at different concentrations in triplicate starting from 0.25 to 5 μM. Said test is used to evaluate the ability of said compounds to inhibit binding of SARS-COV-2 Spike RBD to immobilized human ACE2 using ELISA. IC₅₀ values were computed using four-parameter variable slope sigmoidal dose-response models using Graph Pad Prism 8.0 software.

FIGS. 4A-K show a method of determining cytotoxicity of the present invention, as applied to five drug-like compounds. Specifically. FIGS. 4A-K show MTT assays assessing the viability of HEK293T-hACE2 cells in the presence of an indicated concentration of the compound. FIG. 4F and FIG. 4G show the measurement of cytotoxicity of the compounds of formula (Ia) and formula (IIa), respectively, in Vero-STAT1 KO cells in the presence of an indicated concentration of the compounds. FIG. 4H and FIG. 4I show measurement of cytotoxicity of formula (Ia) and formula (IIa), respectively, in UNCN1T cells in the presence of an indicated concentration of the compounds. FIG. 4J and FIG. 4K show measurement of cytotoxicity of formula (Ia) and formula (IIa), respectively, in Calu-3 cells in the presence of an indicated concentration of the compounds. In HEK293T-hACE2 cells, all five compounds have CC₅₀ values above 100 μM, including formula I (FIG. 4A), formula (IIa) (FIG. 4B), formula V (FIG. 4C), formula VI (FIG. 4D), and formula VII (FIG. 4E).

FIGS. 5A-D show the screening of entry inhibition potential of five drug-like compounds using a pseudovirus assay of the present invention. Specifically. FIGS. 5A-E show HEK-293T-hACE2 cells pretreated with the indicated concentration of compounds and then inoculated with pseudotyped lentiviral particles expressing Spike glycoprotein of SARS-COV-2. The compounds include formula I (FIG. 5A), formula (II) (FIG. 5B), formula V (FIG. 5C), formula VI (FIG. 5D), and formula (VII) (FIG. 5E). At 48 h post-transduction, pseudotype entry was analyzed after normalization against untreated cells by determining luciferase activity in cell lysates. FIG. 5F shows that the percentage inhibition of entry of pseudotyped lentiviral particles expressing Spike glycoprotein of SARS-COV-2 was measured for formula (IIa) at indicated the concentration of the compounds. Notably, cells that received DMSO were considered vehicle controls. The IC₅₀ value was computed using four-parameter variable slope sigmoidal dose-response models using Graph Pad Prism 8.0 software.

FIGS. 6A-D depict SARS-COV-2 dose-response curves for formula (Ia) and formula (IIa) treated and SARS-COV-2 infected UNCN1T and Vero-STAT1 knockout cells, according to a method of the present invention. FIGS. 6A-B depict a formula (Ia) (in blue) and formula (IIa) (in green) dose-response curve by percentage inhibition of SARS-COV-2 replication 24 hpi (FIG. 6A) and 48 hpi (FIG. 6B) in UNCN1T cells with indicated drug concentrations. FIGS. 6C-D show a formula (Ia) (in blue) and formula (IIa) (in green) dose-response curve by percentage inhibition of SARS-COV-2 replication at 24 hpi (FIG. 6C) and 48 hpi (FIG. 6D) in Vero-STAT1 knockout cells with indicated compound concentrations. In embodiments, formula (II)(e.g., formula (IIa)) exhibits a lower IC₅₀ value in FIGS. 6B-6D at all time points, consistent with its enhanced pharmacokinetic profile.

FIGS. 7A-D depict a SARS-COV-2 dose-response curve for formula (Ia) and formula (IIa) treated and SARS-COV-2 variant of concern in infected Calu-3 cells. FIG. 7A shows a formula (Ia) dose-response curve by percentage inhibition of SARS-COV-2 replication 24 hpi in Calu-3 cells infected with South Africa variant (linage: B.1.351) with indicated drug concentrations. FIG. 7B shows a formula (Ia) dose-response curve by percentage inhibition of SARS-COV-2 replication 24 hpi in Calu-3 cells infected with Scotland variant (linage: B.1.222) with indicated drug concentrations. FIG. 7C shows a formula (IIa) dose-response curve by percentage inhibition of SARS-COV-2 replication 24 hpi in Calu-3 cells infected with South Africa variant (linage: B.1.351) with indicated drug concentrations. FIG. 7D shows a formula (IIa) dose-response curve by percentage inhibition of SARS-COV-2 replication 24 hpi in Calu-3 cells infected with Scotland variant (linage: B.1.222) with indicated drug concentrations.

FIG. 8A-B show the impact of time addition of the compounds on replication of SARS-CoV-2 in Vero-STAT1 knockout cells, according to a method of the present invention. FIG. 8A is experimental outline describing the time of adding formula (Ia) and formula (IIa) to the cells. SARS-COV-2 infection, and measurement of viral replication kinetics at the termination of the experiment. FIG. 8B shows the percentage of SARS-COV-2 replication in the presence of vehicle control (DMSO), formula (Ia) (5 μM) and formula (IIa) (5 μM) at −2 hpi. +0) hpi and +4 hpi in Vero-STAT1 knockout cells, respectively. Said observation indicates that these compounds may serve as an effective prophylactic against SARS-COV-2.

FIG. 9A-D depict the combinational effect of remdesivir (RDV) and formula (Ia) treatment against SARS-COV-2 infected UNCN1T cells at 24 h post-infection. FIG. 9A is a dose response curve of remdesivir in SARS-COV-2 infected UNCN1T cells at 24 hpi in the presence of different fixed concentrations of formula (Ia). FIG. 9B shows a dose-response curve of formula (Ia) in SARS-COV-2 infected UNCN1T cells at 24 hpi in the presence of a different fixed concentration of remdesivir. FIG. 9C shows a dose-response percent inhibition matrix of single and combined treatment of remdesivir and formula (Ia) in SARS-COV-2 infected UNCN1T cells at 24 hpi, plotting concentrations of RDV (micromolar) against concentration of formula (Ia) (micromolar). FIG. 9D depicts a 3-D interaction landscape between remdesivir and formula (IIa) calculated based on Loewe additive model using Synergy Finder v.2 in SARS-CoV-2 infected UNCN1T cells at 24 hpi (Loewe synergy score −30.69; with most synergistic area score of −21.34). The graph plots concentrations of RDV (micromolar) against concentrations of formula (Ia) (micromolar) in a 3-D interaction landscape using SynerFinder v.2.

FIGS. 10A-D show the combinational effect of remdesivir and formula (IIa) treatment against SARS-COV-2 infected UNCN1T cells at 24 h post-infection. FIG. 10A is a dose-response curve of remdesivir in SARS-COV-2 infected UNCN1T cells at 24 hpi in the presence of a different fixed concentration of formula (IIa). FIG. 10B is a dose-response curve of formula (IIa) in SARS-COV-2 infected UNCN1T cells at 24 hpi in the presence of a different fixed concentration of remdesivir. FIG. 10C is a dose-response percent inhibition matrix of single and combined treatment of remdesivir and formula (IIa) in SARS-COV-2 infected UNCN1T cells at 24 hpi, plotting concentrations of RDV (micromolar) against concentration of formula (IIa) (micromolar). FIG. 10D shows an interaction landscape between remdesivir and formula (IIa) calculated based on Loewe additive model using Synergy Finder v.2 in SARS-COV-2 infected UNCN1T cells at 24 hpi (Loewe synergy score 26.63: with most synergistic area score of 37.25). The graph plots concentrations of RDV (micromolar) against concentrations of formula (IIa) (micromolar) in a 3-D interaction landscape using SynerFinder v.2.

FIGS. 11A-B show binding affinities in a microscale thermophoresis assay (MST), demonstrating that formula (IIa) does not bind to S-RBD along, nor ACE2 alone. Rather, formula (IIa) binds to an S-RBD/ACE2 complex. The microscale thermophoresis assay was also used to derive binding kinetics for formula (IIa)/Hu-1 S-RBD only (FIG. 11A) and formula (IIa)/ACE2 only (FIG. 11B). The formula (IIa)/ACE2 only binding curve (FIG. 11B) shows a K_{d} of 3.7 micromolar.

FIGS. 12A-D depict a microscale thermophoresis (“MST”) assay analyzing binding of formula (IIa) and formula (IIb) compounds with WT (“Hu-1”) and Delta S-RBD/ACE2 complexes. The MST assay with formula (IIa) and Hu-1 (FIG. 12A) provides a K_{d} of 299 nM. The MST assay with formula (IIa) and Delta (FIG. 12B) provides a K_{d} of 200 nM. The MST assay with formula (IIb) and Hu-1 (FIG. 12C) provides a K_{d} of 31 nM. The MST assay with formula (IIb) and Delta (FIG. 12D) provides a K_{d} of 90 nM. Thus, formula (IIa) exhibits about a 10-fold decreased binding affinity with Hu-1 S-RBD/ACE complex and about a 10-fold decreased binding affinity with Delta S-RBD/ACE complex relative to its derivative formula (IIb).

FIGS. 13A-13D show changes in sidechain conformation upon binding of the compounds of formula (Ia) and formula (IIa) to an interface of SARS-COV-2 spike protein receptor binding domain (RBD) and a host cell ACE-2 receptor (“ACE2:Spike RBD”). Changes in conformation were determined by induced-fit docking of compounds formula (Ia) and formula (IIa). All sidechains shown display significant changes upon induced-fit docking (IFD) of formula (Ia) or formula (IIa). FIG. 13A shows conformational changes upon docking of the compound of formula (Ia) to the interface of ACE2:Spike RBD. Yellow carbons represent the conformation of S-RBD sidechains before IFD, whereas cyan carbon correspond to the sidechain conformation of S-RBD after IFD. FIG. 13B shows the mode of formula (Ia) binding before (teal carbons) and after IFD (magenta carbons) docking to ACE2:Spike RBD. FIG. 13C shows a conformational change upon docking of formula (IIa). FIG. 13D shows the binding mode of formula (II) binding before (teal carbons) and after IFD (magenta carbons).

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention. An artisan of ordinary skill in the art need not view, within isolated figure(s), the near infinite number of distinct permutations of features described in the following detailed description to facilitate an understanding of the present invention.

### DETAILED DESCRIPTION

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

### I. Definitions and Interpretation

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. The nomenclature used in connection with, and the laboratory procedures and techniques of, analytical chemistry, synthetic organic chemistry, and medicinal and pharmaceutical chemistry described herein are those well-known and commonly used in the art.

As used in the description and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, the term “alkyl” or “alkyl groups” refers to saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (e.g., isopropyl, tert-butyl, sec-butyl, isobutyl, etc.), and alkyl-substituted alkyl groups (e.g., alkyl-substituted cycloalkyl groups and cycloalkyl-substituted alkyl groups).

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxy carbonyloxy, carboxylate, alkylcarbonyl, arylcarbonyl, alkoxy carbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, the term “about” will be understood by persons of ordinary skill in the art and will vary to some extent depending upon the context in which it is used. If there are uses of the term which are not clear to persons of ordinary skill in the art given the context in which it is used. “about” will mean up to plus or minus 10% of the particular term.

Numeric ranges recited within the specification, including ranges of “at least”. “greater than.” or “less than” a numeric value, are inclusive of the numbers defining the range and include each integer within the defined range. Ranges may be expressed as from “about” one particular value, and/or to “about” another particular value. When such a range is expressed, another aspect includes from the one particular value and/or to the other particular value. Similarly, when values are expressed as approximations, by use of the antecedent “about.” it will be understood that the particular value forms another aspect. It will be further understood that the endpoints of each of the ranges are significant both in relation to the other endpoint, and independently of the other endpoint.

The term “administering” an active agent should be understood to mean providing an active agent to the subject in need of treatment in a form that can be introduced into that individual's body in a therapeutically useful form and therapeutically effective amount. As used herein. “administering” further refers to the introduction of an agent, such as a disclosed entry inhibitor, into a subject by a chosen route. Administration can be local or systemic. For example, if the chosen route is intranasal, the agent (such as an immunogen comprising a recombinant coronavirus S ectodomain trimer stabilized in a prefusion conformation) is administered by introducing the composition into the nasal passages of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

As used herein, an “adjuvant” refers to a vehicle used to enhance antigenicity. In some embodiments, an adjuvant can include a suspension of minerals (alum, aluminum hydroxide, or phosphate) on which antigen is adsorbed: or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some embodiments, the adjuvant used in a disclosed pharmaceutical composition is a combination of lecithin and carbomer homopolymer (such as the ADJUPLEX™ adjuvant available from Advanced BioAdjuvants. LLC, see also Wegmann. Clin Vaccine Immunol. 22(9): 1004-1012, 2015). Additional adjuvants for use in the disclosed immunogenic compositions include the QS21 purified plant extract. Matrix M. ASO1. MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2. RANTES. GM-CSF. TNF-α. IFN-γ. G-CSF. LFA-3, CD72. B7-1. B7-2. OX-40L. 4-1BBL and toll-like receptor (TLR) agonists, such as TLR-9 agonists. Additional description of adjuvants can be found, for example, in Singh (ed.) Vaccine Adjuvants and Delivery Systems. Wiley-Interscience. 2007). Adjuvants can be used in combination with the disclosed immunogens.

As used herein. “antiviral agents” may include drugs approved by the Food and Drug Administration (FDA) for the treatment or control of viral infections. In the context of Covid-19, antivirals may include the small molecule pharmaceutical compositions disclosed herein (e.g., small molecule entry inhibitors), remdesivir, lagevrio (molnupiravir), paxlovid (ritonavir), and the compounds like these. Mechanistically, antiviral agents primarily target stages in the viral life cycle. Exemplar target stages in the viral life cycle include: viral attachment to host cell, uncoating, synthesis of viral mRNA, translation of mRNA, replication of viral RNA and DNA, maturation of new viral proteins, budding, and release of newly synthesized virus.

As used herein, an “amino acid substitution” refers to the replacement of one amino acid in a polypeptide with a different amino acid.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “Coronaviridae” refers to a family of enveloped, positive-sense, single-stranded RNA viruses. Viruses currently known to infect humans from the coronavirus family are from the alphacoronavirus and betacoronavirus genera. Additionally, it is believed that the gammacoronavirus and deltacoronavirus genera may infect humans in the future. The term “coronaviruses” refers to any virus in the Coronaviridae family, including, without limitation. Middle East Respiratory Syndrome (MERS) coronavirus, Human coronavirus 229E (HCoV-229E). Human coronavirus OC43 (HCoV-OC43). Severe Acute Respiratory Syndrome-related coronavirus (SARS-COV: also referred to as SARS-COV-1). Human coronavirus NL63 (HCoV-NL63. New Haven coronavirus). Human coronavirus HKU1, novel coronavirus (2019-nCOV), also known as Severe Acute Respiratory Syndrome coronavirus 2 (SARS-COV-2), which is the causal agent of the disease known as Wuhan pneumonia or coronavirus disease 2019 (COVID-19), and related strains of any of the coronaviruses. The term “SARS-COV-2” may be used interchangeably with the term “Wuhan coronavirus” and variations thereof throughout the disclosure. The term coronavirus and variations thereof are used interchangeably throughout the disclosure. Other Coronaviridae viruses are used as examples, targets and standards by which the presently disclosed compounds are measured, including, without limitation, MERS (Middle East Respiratory Syndrome) coronavirus. A non-limiting example of a deltacoronaviruses is the Swine Delta Coronavirus (SDCV). The viral genome is capped, polyadenylated, and covered with nucleocapsid proteins. The coronavirus virion includes a viral envelope containing type I fusion glycoproteins referred to as the spike (S) protein. Most coronaviruses have a common genome organization with the replicase gene included in the 5′-portion of the genome, and structural genes included in the 3′-portion of the genome.

As used herein, the term “Coronavirus Spike (S) protein” refers to a class I fusion glycoprotein initially synthesized as a precursor protein. Individual precursor S polypeptides form a homotrimer and undergo glycosylation within the Golgi apparatus as well as processing to remove the signal peptide, and cleavage by a cellular protease to generate separate 51 and S2 polypeptide chains, which remain associated as S1/S2 protomers within the homotrimer and is therefore a trimer of heterodimers. The 51 subunit is distal to the virus membrane and contains the receptor-binding domain (RBD) that mediates virus attachment to its host receptor. The S2 subunit contains fusion protein machinery, such as the fusion peptide, two heptad-repeat sequences (HR1 and HR2) and a central helix typical of fusion glycoproteins, a transmembrane domain, and the cytosolic tail domain.

As used herein. “condition” or “health condition” refers to an ex vivo, in vivo, or in cellulo state of a subject or organism. A health condition may relate to, for example, the presence of health-related viruses in a given location. In the present context, the claims contemplate modulation of cell surface interactions with a small molecule inhibitor in order to inhibit the replication of a virus. The range of subject animal species that may suffer from a health condition is also very broad, including humans, domesticated animals, farm animals, aquatic invertebrates, and the like.

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

As used herein. “disease” refers to a condition of a living animal or plant body or of one of its parts that impairs normal functioning and is typically manifested by distinguishing signs and symptoms. Diseases may include bacterial infections, viral infections, resistant viral and bacterial infections, genetic disorders, cancers, any conditions that involve a copper homeostatic component, and other harmful health conditions known in the art.

As used herein, an “entry inhibitors” (also referred to as “fusion inhibitors”), are a class of antiviral drug that prevent a virus from entering a cell, for example, by blocking a cell surface receptor. Entry inhibitors may comprise small molecules (e.g. the small molecule inhibitors of the present invention), antibodies, and the like.

As used herein, the term “exemplary” refers to an example, an instance, or an illustration, and does not indicate a most preferred embodiment unless otherwise stated.

As used herein. “inhibition” refers to the interruption of a chemical pathway owing to one chemical substance inhibiting the effect of another by competing with it for binding or bonding (e.g. “competitive inhibition”). As used herein. “inhibiting or treating a disease” refers to inhibiting the full development of a disease or condition, for example, in a subject who is at risk for a disease such as a CoV infection. This may be accomplished by inhibiting replication of a virus with small molecule entry inhibitors. “Treatment” refers to a therapeutic intervention that ameliorates a sign or symptom of a disease or pathological condition after it has begun to develop. The term “ameliorating.” with reference to a disease or pathological condition, refers to any observable beneficial effect of the treatment.

Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

As used herein, a “pharmaceutically acceptable carrier” refers to the pharmaceutically acceptable carriers of use that are conventional. Pharmaceutically acceptable carriers that may be used in these compositions (e.g., compounds I, II, or III) include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as small molecule entry inhibitors) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

As used herein, a “polypeptide” refers to any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

As used herein. “therapeutically effective amount” comprises a pharmaceutically effective amount, and refers a concentration and/or volume of compound which is effective in reducing, eliminating, treating, preventing or controlling the symptoms of the infections, diseases, disorders, or other conditions resulting from infection with SARS-COV-1 virus, SARS-CoV-2 virus, and the like. A pharmaceutically effective amount, in the context of a SARS-COV-1 and SARS-COV-2 infection, refers to the amount administered so as to maintain an amount which suppresses or inhibits circulating virus throughout the period during which infection is evidenced such as by presence of anti-viral antibodies, presence of culturable virus and presence of viral antigen in patient sera, or symptoms that are identifiable by a medical professional.

As used herein, a “mutation” refers to a single change in a virus's genome (genetic code). Mutations happen frequently, but only sometimes change the characteristics of the virus. In some cases they are used in combination therapy for the treatment of an infection (e.g. SARS-CoV-2. HIV, hepatitis D, and the like).

As used herein, a “nucleoside analogue(s)” refers to nucleosides which contain a nucleic acid analogue, a sugar, and a phosphate groups with one to three phosphates. As an antiviral, they are generally used to prevent viral replication in infected cells. Remdesivir or ribavirin is an example of a nucleoside analogue applicable in the context of SARS-COV treatment. Nucleotide and nucleoside analogues can also be found naturally. Examples include ddhCTP (3′-deoxy-3′,4′didehydro-CTP) produced by the human antiviral protein viperin and sinefungin (a S-Adenosyl methionine analogue) produced by some Streptomyces.

As used herein. “variant” refers to a viral genome that may contain one or more mutations. In some cases, a group of variants with similar genetic changes, such as a lineage or group of lineages, may be designated by public health organizations as a Variant of Concern (VOC) or a Variant of Interest (VOI) due to shared attributes and characteristics that may require public health action. As used herein “variant” comprises VOCs. VOIs, and Variant Being Monitored (VBMs). In the case of SARS-COV, a VOC is a variant for which there is evidence of an increase in transmissibility, more severe disease (for example, increased hospitalizations or deaths), significant reduction in neutralization by antibodies generated during previous infection or vaccination, reduced effectiveness of treatments or vaccines, or diagnostic detection failures. A VOI is a variant with specific genetic markers that have been associated with changes to receptor binding, reduced neutralization by antibodies generated against previous infection or vaccination, reduced efficacy of treatments, potential diagnostic impact, or predicted increase in transmissibility or disease. VBMs include those where data indicates there is a potential or clear impact on approved or authorized medical countermeasures or that have been associated with more severe disease or increased transmission but are no longer detected, or are circulating at very low levels, in the United States. These variants do not pose a significant and imminent risk to public health in the United States.

Homologs and variants of a polypeptide (such as a coronavirus RBD) are typically characterized by possession of at least about 75%, for example at least about 80%. 85%. 90%. 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet.

As used herein. “subject” refers to a living multi-cellular vertebrate organism, a category that includes human and non-human mammals, such as non-human primates, pigs, camels, bats, sheep, cows, dogs, cats, rodents, and the like. In an example, a subject is a mammal (e.g. a human, aquatic mammal, wild animal, or the like). The subject can be a domestic animal (such as a dog or a cat) or a farm animal (such as a cow or a pig). In an additional example, a subject is selected that is in need of inhibiting a coronavirus infection, such as a SARS-COV or MERS-CoV infection, or inhibiting replication of a coronavirus. For example, the subject is either uninfected and at risk of the coronavirus infection or is infected and in need of treatment.

As used herein. “variant” refers to the South African variant. Scotland variant, and other variants including Variant of Interest (VOIs). Variants Being Monitored (VBMs), and Variants of Concern (VOC). A VOC is a variant for which there is evidence of an increase in transmissibility, more severe disease, significant reduction in neutralization by antibodies generated during previous infection or vaccination, reduced effectiveness of treatments or vaccines, or diagnostic detection failures. A VOI is a variant with specific genetic markers that have been associated with changes to receptor binding, reduced neutralization by antibodies generated against previous infection or vaccination, reduced efficacy of treatments, potential diagnostic impact, or predicted increase in transmissibility or disease. VBMs include those cases where data indicates there is a potential or clear impact on approved or authorized medical countermeasures or that have been associated with more severe disease or increased transmission but are no longer detected, or are circulating at very low levels, in the United States. These variants do not pose a significant and imminent risk to public health in the United States.

Exemplar SARS-COV-2 variant lineages include: Alpha (B.1.1.7 and Q lineages). Beta (B.1.351 and descendent lineages). Gamma (P.1 and descendent lineages). Epsilon (B.1.427 and B.1.429). Eta (B.1.525), Iota (B.1.526). Kappa (B.1.617.1), variant 1.617.3. Mu (B.1.621. B.1.621.1). Zeta (P.2), and the like. In embodiments, in the context of SARS-COV-2. VOCs include B. 1.1.7 and Q lineages (Dec. 29, 2020). B.1.351 and descendent lineages (Dec. 29, 2020). P.1 and descendent lineages (Dec. 29, 2020), and B.1.427/B.1.429 (Mar. 19, 2021). Examples of VOIs include and B.1.427 and B. 1.429 (Feb. 26, 2021; Jun. 29, 2021). B.1.525 (Feb. 26, 2021). B. 1.526 (Feb. 26, 2021). B. 1.617.1 (May 7, 2021). B.1.617.3 (May 7, 2021), and P.2 (Feb. 26, 2021). Examples of VBMs include B.1.1.7 and Q lineages (Sep. 21, 2021). B.1.351 and descendent lineages (Sep. 21, 2021). P.1 and descendent lineages (Sep. 21, 2021). B. 1.427 and B.1.429 (Sep. 21, 2021). B.1.427/B.1.429 (Sep. 21, 2021). B.1.525 (Sep. 21, 2021). B.1.526 (Sep. 21, 2021). B.1.617.1 (Sep. 21, 2021). B.1.617.3 (Sep. 21, 2021), and P.2 (Sep. 21, 2021).

### II. Compounds

The term “compound” or “candidate compound” used herein describes any molecule, either naturally occurring or synthetic that may be tested in an assay, such as a screening assay, or specifically in the method for identifying a compound capable of binding and preventing replication and/or infection of SARS-COV-2 and/or SARS-COV-1. As such, these compounds comprise organic and inorganic compounds. The compounds may be small molecules or chemicals in the preferred embodiments. In other embodiments, compounds may include peptides, antibodies or ISVDs or active antibody fragments.

Compounds of the present invention include both those designed or identified using a screening method of the invention and those which are capable of binding and neutralizing SARS-COV-2 and/or SARS-COV-1. Compounds capable of binding and neutralizing SARS-CoV-2, for example, may be produced using a CADD-based approach in addition to screening methods based on use of the atomic coordinates corresponding to the 3D structure of the S-RBD/Spike Complex and/or the S-RBD/Spike Complex bound to ACE2 (also referred to herein as “ACE2:Spike RBD”) as presented herein. The candidate compounds and/or compounds identified or designed using a method of the present invention may be any suitable compound, synthetic or naturally occurring, preferably synthetic. In one embodiment, a synthetic compound selected or designed by the methods of the invention preferably has a molecular weight equal to or less than about 5000, 4000, 3000, 2000, 1000 or more preferably less than about 500 Daltons. A compound of the present invention is preferably soluble under physiological conditions. Such compounds can comprise functional groups necessary for structural interaction with proteins, particularly hydrogen bonding, and typically include at least an amine, carbonyl, hydroxyl or carboxyl group, preferably at least two of the functional chemical groups. The compound may comprise cyclical carbon or heterocyclic structures and/or aromatic or polyaromatic structures substituted with one or more of the above functional groups. Compounds can also comprise biomolecules including peptides, saccharides, fatty acids, steroids, purines, pyrimidines, derivatives, structural analogues, or combinations thereof.

### A. Screening Compounds Using CADD Approach

FIG. 1A shows the computer-aided process for screening entry inhibitor compounds. The process comprises the screening of at least 8 million compounds using a computer-aided drug design (CADD) approach. In some embodiments, between 5 million and 15 million compounds are screened using the CADD approach. In embodiments, the residues found at the interface of S-RBD and ACE2 are summarized described in Table 1. Residues at the interface of S-RBD and ACE2 are also shown in FIG. 1B. Further, FIG. 1C shows a zoomed-in view of the binding pocket.

TABLE 1
Residues at the interface of S-RBD and ACE2

| S-RBD residues | ACE2 residues |
|---|---|
| K417, V445, G446, Y449, Y453, | S19, Q24, T27, F28, D30, K31, H34, |
| L455, F456, Y473, A475, G476, | E35, E37, D38, Y41, Q42, L45, L79, |
| F486, Y489, Q493, Q498, T500, | M82, Y83, K353, G354, D355 and |
| N501, G502, V503, and Y505 | R357 |

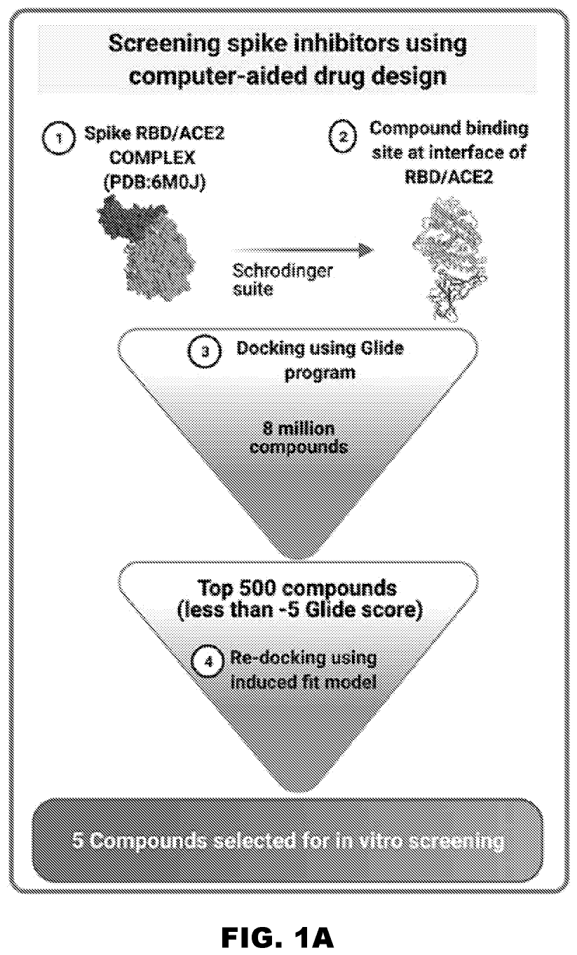

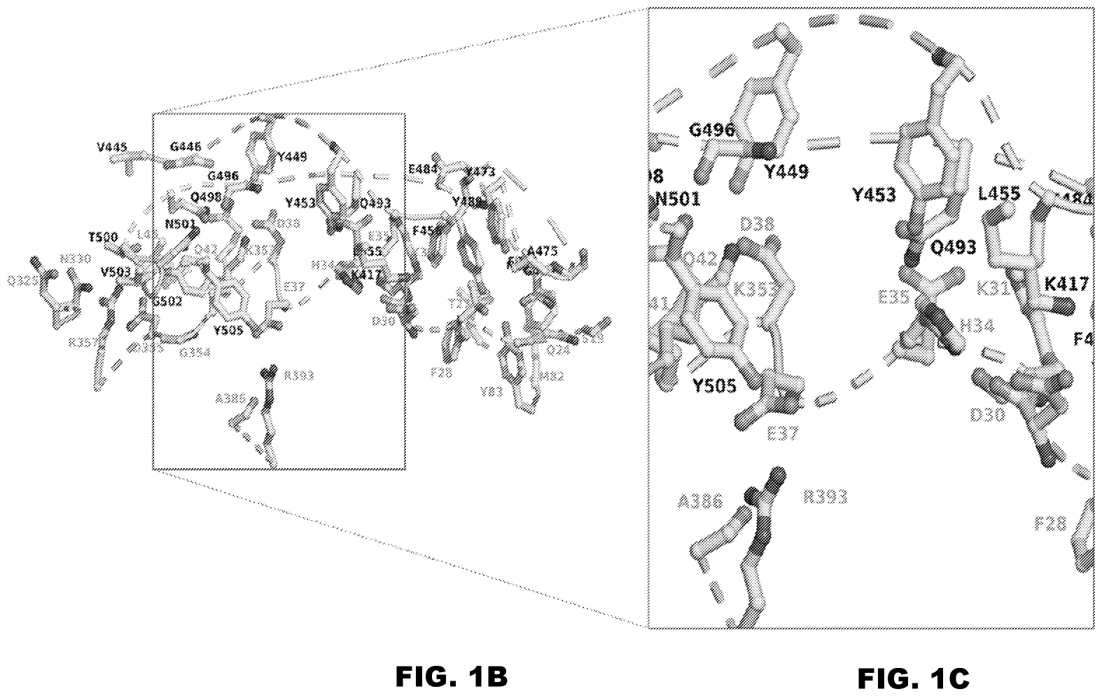

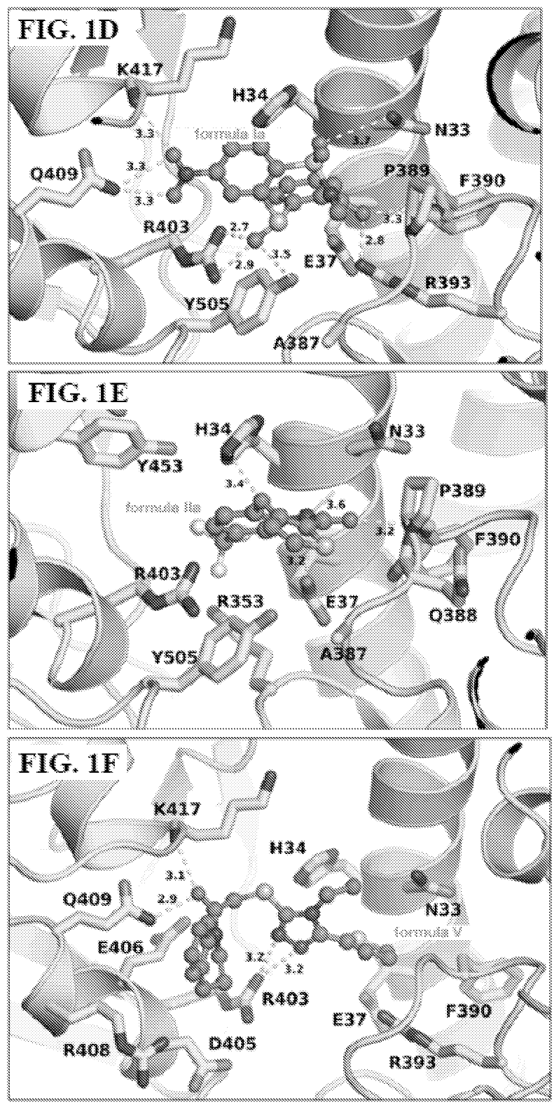

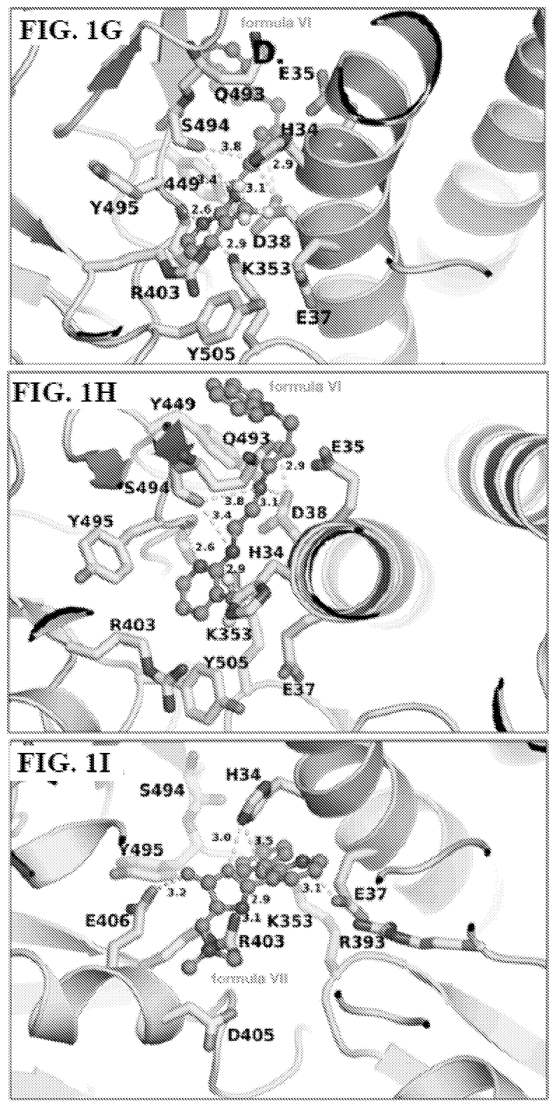

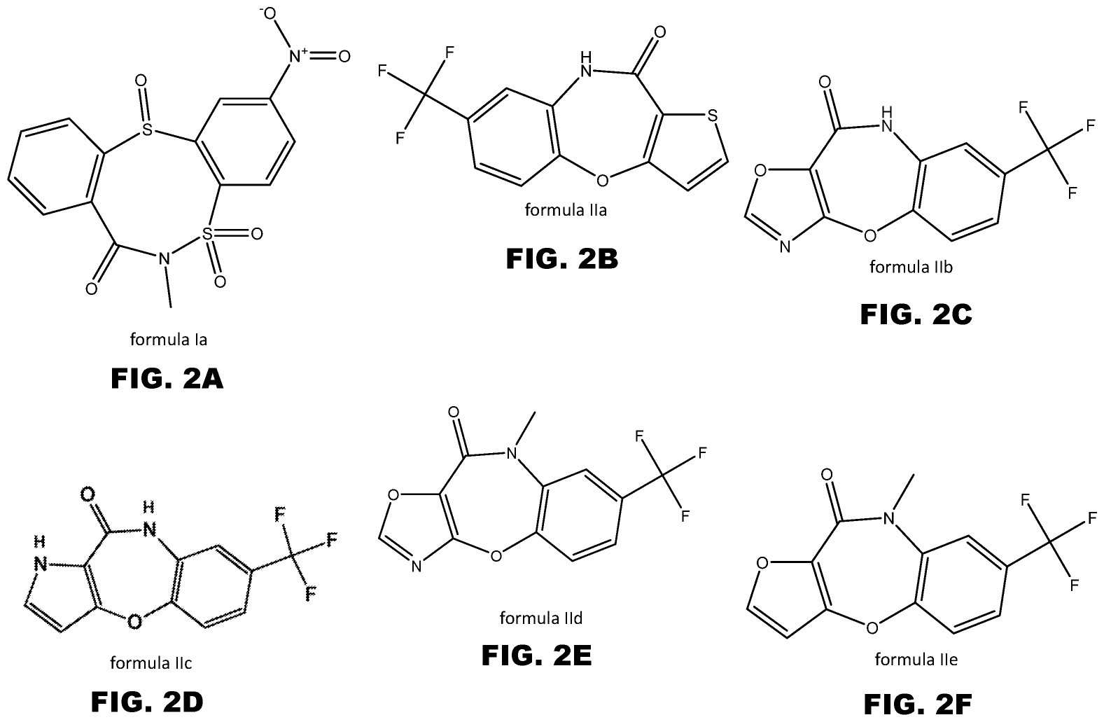

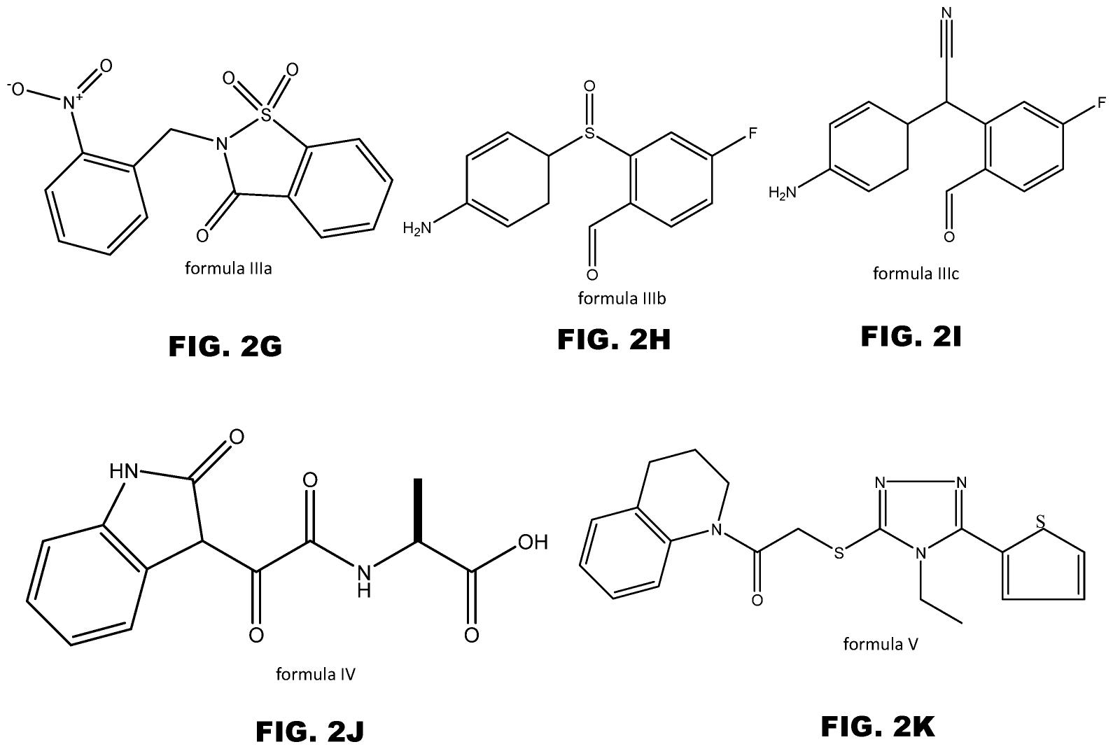

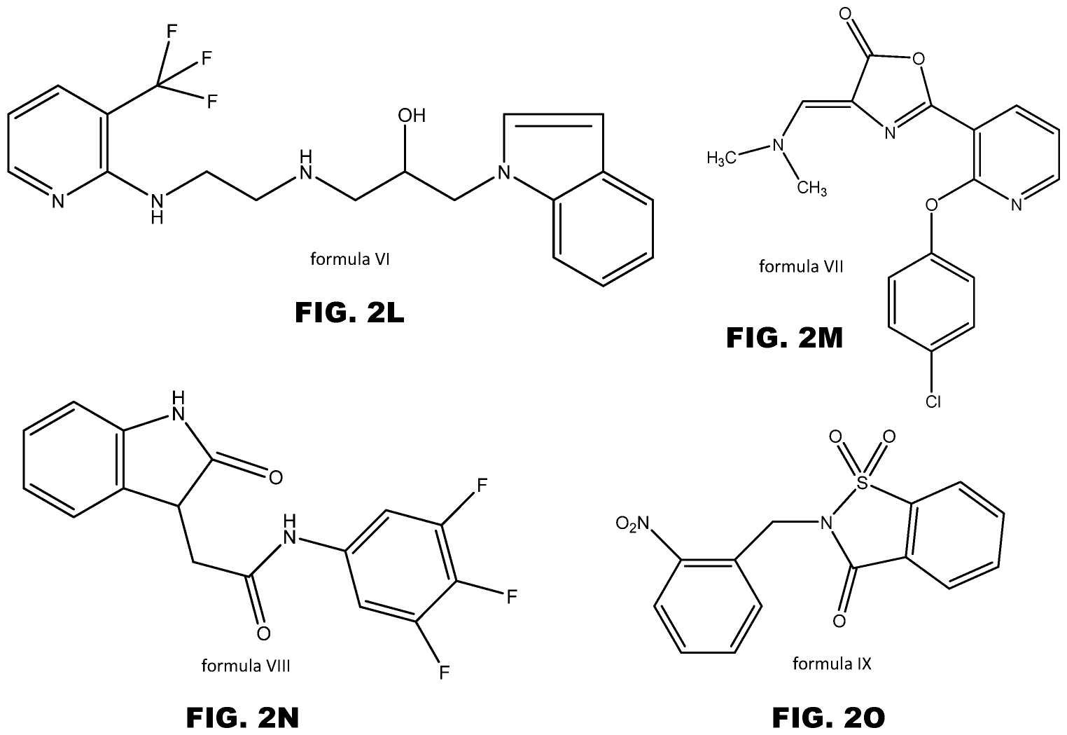

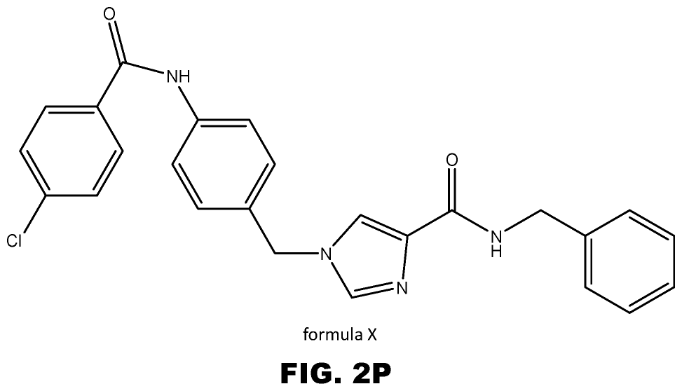

In embodiments, a method for screening entry inhibitors is provided wherein, in a first step, docking scores and binding geometries of candidate compounds are analyzed. In a second step, said compounds are tested for antiviral activity in the cell-based assays. Finally, said compounds may be derivatized to improve pharmacokinetics. This method provides a standard approach for the selection of compounds based on docking scores and visual inspection of their binding geometries in the binding pocket formed by S-RBD/Spike Complex and host ACE2 (also referred to herein as, “ACE2:Spike RBD”). Exemplary compounds selected are shown in FIG. 1D-I, including formula (Ia)(FIG. 1D), formula (IIa)(FIG. 1E), formula (V)(FIG. 1F), formula (VI)(FIGS. 1G & FIG. 1H), and formula (VII) (FIG. 1I). After testing the compounds for antiviral activity, formula (Ia) and formula (IIa) were identified as drug-like compounds. Later, formula (IIa) was derivatized to produce formula (IIb) (FIG. 2C), formula (IIc) (FIG. 2D), formula (IId) (FIG. 2E), and formula (IIe) (FIG. 2F). As described above, activity assays show that formula (IIb) has improved binding kinetics relative to formula (IIa) with both Hu-1 and Delta. Additional compounds tested for antiviral activity and/or considered for future derivatization include formula (IIIa)(FIG. 2G), formula (IIIb)(FIG. 2H), formula (IIIc)(FIG. 2I), formula (IV)(FIG. 2J), formula (V)(FIG. 2K), formula (VI)(FIG. 2L), formula (VII)(FIG. 2M), formula (VIII)(FIG. 2N), formula (IX)(FIG. 2O), and formula (X)(FIG. 2P). Properties of several of the above compounds are summarized in Table 2 below, including compound ID, IUPAC Name, and Structure.

TABLE 2
Entry inhibitor compounds and derivatives

| Compound: | FIG. | IUPAC Names: | Structure: |
|---|---|---|---|
| formula (Ia) | 2A | 6-methyl-2-nitrodibenzo[d,g]- [1,6,2]dithiazocin-7(6H)- one 5,5, 12-trioxide | 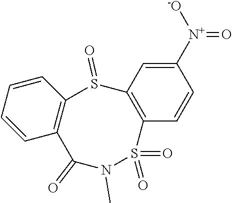 |
| formula (IIa) | 2B | 7-(trifluoromethyl)benzo[b]- thieno[2,3-f][1,4]oxazepin- 10(9H)-one | 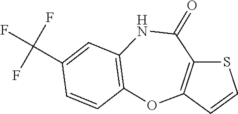 [SMILES: O=C1Nc2cc(C(F)(F)F)ccc2Oc2ccsc21] |
| formula (IIb) | 2C | 7-(trifluoromethyl)[1,3]- oxazolo[4,5-b][1,5]benzox- azepin-10(9H)-one | 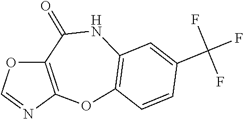 [SMILES: O=C1Nc2cc(C(F)(F)F)ccc2Oc2ncoc21] |
| formula (IIc) | 2D | 7-(trifluoromethyl)-1H- pyrrolo[3,2-b][1,5]benzox- azepin-10(9H)-one | 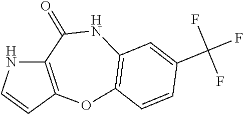 [SMILES: O=C1Nc2cc(C(F)(F)F)ccc2Oc2cc[nH]c21] |
| formula (IId) | 2E | 9-methyl-7-(trifluoromethyl)- [1,3]oxazolo[4,5-][1,5]- benzoxazepin-10(9H)-one | 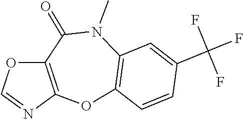 [SMILES: CN1C(=O)c2ocnc2Oc2ccc(C(F)(F)F)cc21] |
| formula (IIe) | 2F | 9-methyl-7-(trifluoromethyl)- furo[3,2-b][1,5]benzox- azepin-10(9H)-one | 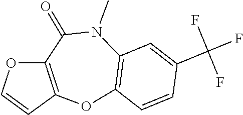 [SMILES: CN1C(=O)c2occc2Oc2ccc(C(F)(F)F)cc21] |
| formula (IIIa) | 2G | 1-(morpholin-4-yl)-2- [(1,2,4]triazolo[3,4-c][1,2,4]- benzotriazin-1-yl)sulfanyl]- ethan-1-one | 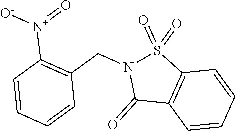 [SMILES: O=C1c2ccccc2S(=O)(=O)N1Cc1ccccc1[N+](=O)[O-]] |
| formula (IIIb) | 2H | O═S(clccc(N)ccl)clcc(F)cccl C═O | 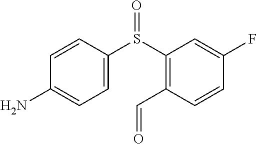 [SMILES: Nc1ccc(S(=O)c2cc(F)ccc2C=O)cc1] |
| formula (IIIc) | 2I | Nclccc(ccl)C(C#N)clcc(F)cccl C═O | 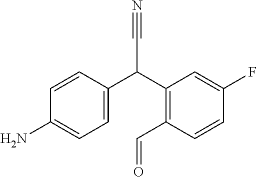 [SMILES: N#CC(c1ccc(N)cc1)c1cc(F)ccc1C=O] |
| formula (IV) | 2J | 2-(2-oxo-2-(2-oxoindolin-3- yl)acetamido)propanoic acid | 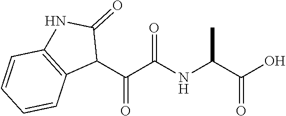 [SMILES: C[C@H](NC(=O)C(=O)C1C(=O)Nc2ccccc21)C(=O)O] |
| formula (V) | 2K | 1-(3,4-dihydroquinolin- 1(2H)-yl)-2-((4-ethyl-5- (thiophen-2-yl)-4H-1,2,4- triazol-3-yl)lhio)elhanone | 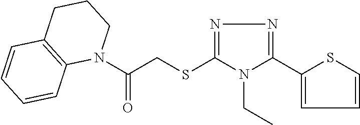 [SMILES: CCn1c(SCC(=O)N2CCCc3ccccc32)nnc1-c1cccs1] |
| formula (VI) | 2L | 1-(1H-indol-1-yl)-3-((2-((3- (trifluoromethyl)pyridin-2- yl)amino)ethyl)amino)- propan-2-ol | 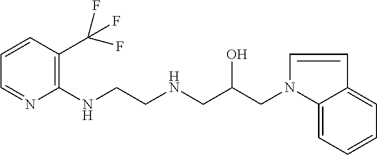 [SMILES: OC(CNCCNc1ncccc1C(F)(F)F)Cn1ccc2ccccc21] |
| formula (VII) | 2M | (Z)-2-(2-(4-chlorophenoxy)- pyridin-3-yl)-4-((dimethyl- amino)methylene)oxazol- 5(4H)-one | 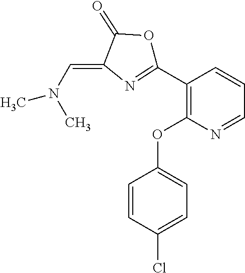 [SMILES: CN(C)/C=C1\N=C(c2cccnc2Oc2ccc(Cl)cc2)OC1=O] |
| formula (VIII) | 2N | 2-(2-oxo-2,3-dihydro-1H- indol-3-yl)-N-(3,4,5- trifluorophenyl)acetamide | 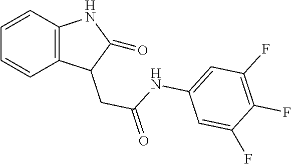 [SMILES: O=C(CC1C(=O)Nc2ccccc21)Nc1cc(F)c(F)c(F)c1] |
| formula (IX) | 2O | 2-[(2-nitrophenyl)methyl]- 1H-1l⁶,2-benzothiazole- 1,1,3(2H)-trione | 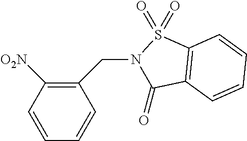 [SMILES: O=C1c2ccccc2S(=O)(=O)N1Cc1ccccc1[N+](=O)[O-]] |
| formula (X) | 2P | N-benzyl-1-[(4-[(4-chloro- benzoyl)amino]phenyl]meth- yl]imidazole-4-carboxamide | 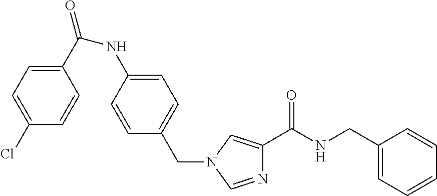 [SMILES: O=C(Nc1ccc(Cn2cnc(C(=O)NCc3ccccc3)c2)cc1)c1ccc(Cl)cc1] |

In embodiments, the docking scores of formula (V), formula (VI), and formula (VII) are −7.6, −7.2, and −6.4, respectively. The best pose (based on the docking score) of formula (Ia) is docked in a pocket formed by S-RBD residues R403, E406, Q409, K417, Y505, and ACE2 residues N33, H34, E37, R393, F390, P389, Q388, A387 (FIG. 1D). In other embodiments, formula (IIa) docks in the same pocket. In still other embodiments, formula (IIa) docks in a pocket formed by Y505, R403, Y453 (from S-RBD), and N33, H34, E37, P389, F390, Q388, and A387 (FIG. 1E). As can be seen from the docking poses of the two compounds shown in FIG. 1D and FIG. 1E, in embodiments formula (Ia) has a more significant number of polar interactions (shown as dotted yellow lines in FIG. 1D) with pocket residues than does formula (IIa), which has only four polar interactions with the pocket residues as shown in FIG. 1E. In embodiments, said limited polar interactions may result in an improved docking score of formula (IIa) compared with formula (Ia). FIG. 1F-1I show that the compounds of formula (V)(FIG. 1F), formula (VI)(FIG. 1G and FIG. 1F), and formula (VII)(FIG. 1I) are adapted to dock in the same pocket as formula (Ia) (FIG. 1D). Therefore, in embodiments, best pose studies show that at least compounds of formula (Ia), formula (IIa), and formula (III) are adapted to bind at the interface of S-RBD (wherein S-RBD is a SARS-COV-1 and/or SARS-COV-2 spike protein receptor binding domain) and a host cell ACE-2 receptor.

### B. Impact of Compounds on Binding of ACE2:Spike RBD of SARS-COV-2

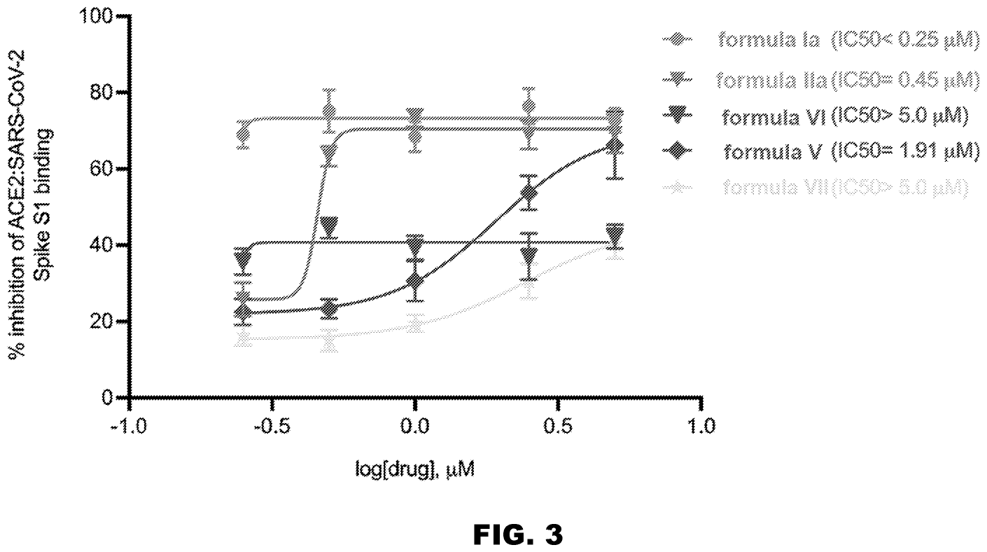

FIG. 3 shows the screening of compounds selected by a computer-aided drug design approach. The present disclosure provides a method for screening a library of compounds in order to identify compounds that inhibit ACE2: SARS-COV-2 Spike RBD (“ACE2:Spike RBD”). Said Spike/ACE2 Inhibitor Screening Assay was carried out as described in the Examples section below. In embodiments, formula (Ia), formula (IIa), formula (V), formula (VI), and formula (VII) are tested at different concentrations, in triplicate, starting from 0.25 to 5 μM. The five compounds were shown to inhibit/block the binding of ACE2 and Spike RBD to different extents. Notably, in embodiments all five compounds are adapted to reverse the binding between ACE2 and Spike RBD in a dose-dependent manner.

In some embodiments, formula (Ia) has an IC₅₀ of at least 0.25 μM. In contrast, formula (IIa), formula (V), formula (VI), and formula (VII) have an IC₅₀ of 0.45, 1.91, >5.0, and >5.0 μM, respectively.

### C. Assessing Binding Affinity Using Microscale Thermophoresis Assays

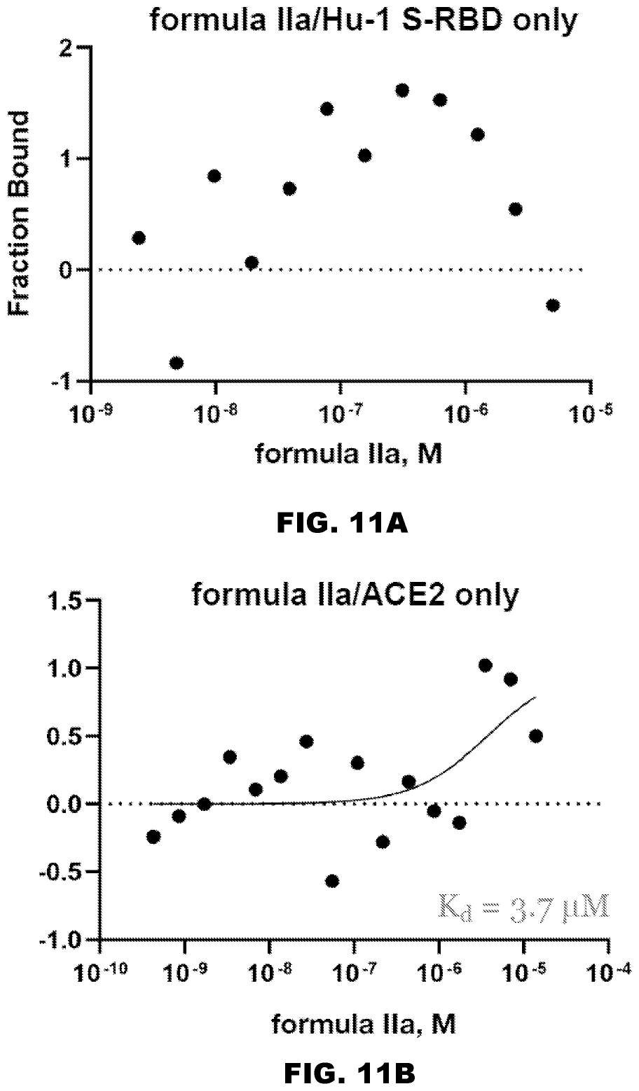

In other embodiments, methods are provided wherein microscale thermophoresis (MST) assays are used to determine whether formula (IIa) and formula (IIb) bind to S-RBD alone (FIG. 11A) and/or ACE2 alone (FIG. 11B). In addition, said MST assays assess whether the compounds bind to the S-RBD/ACE2 complex. In one embodiment, formula (IIa) does not bind to S-RBD (no K_{d} measured) nor ACE2 alone (K_{d}=3.7 μM), but does bind to S-RBD/ACE2 complex (K_{d}=299 nM).

The method further provides for the use of microscale thermophoresis to derive binding kinetics for formula (IIa)/Hu-1 S-RBD only (FIG. 11A) and formula (IIa)/ACE2 only (FIG. 11B). In embodiments, the binding curve for “formula (IIa)/ACE2 only” (FIG. 11B) is used to derive a K_{d} of 3.7 micromolar. In embodiments, formula (IIa) derivatives include formula (IIb) (FIG. 2C), formula (IIc) (FIG. 2D), formula (IId) (FIG. 2E), and formula (IIe) (FIG. 2F). In some embodiments, said formula (IIa) derivatives do not bind to S-RBD and ACE2 alone, but do bind to S-RBD/ACE2 complex. Further to the above, the disclosure generally provides for methods of treating a SARS-COV-2 virus, comprising administering a compound to a subject infected with SARS-COV-2 virus, wherein the compound binds to SARS-COV-2 S-RBD/ACE2 Complex but does not bind to S-RBD alone or ACE-2 alone, wherein the compound comprises formula (II) (e.g., formula (IIa), formula (IIb), formula (IIc), formula (IId), or formula (IIe), or a pharmaceutically acceptable salt thereof.

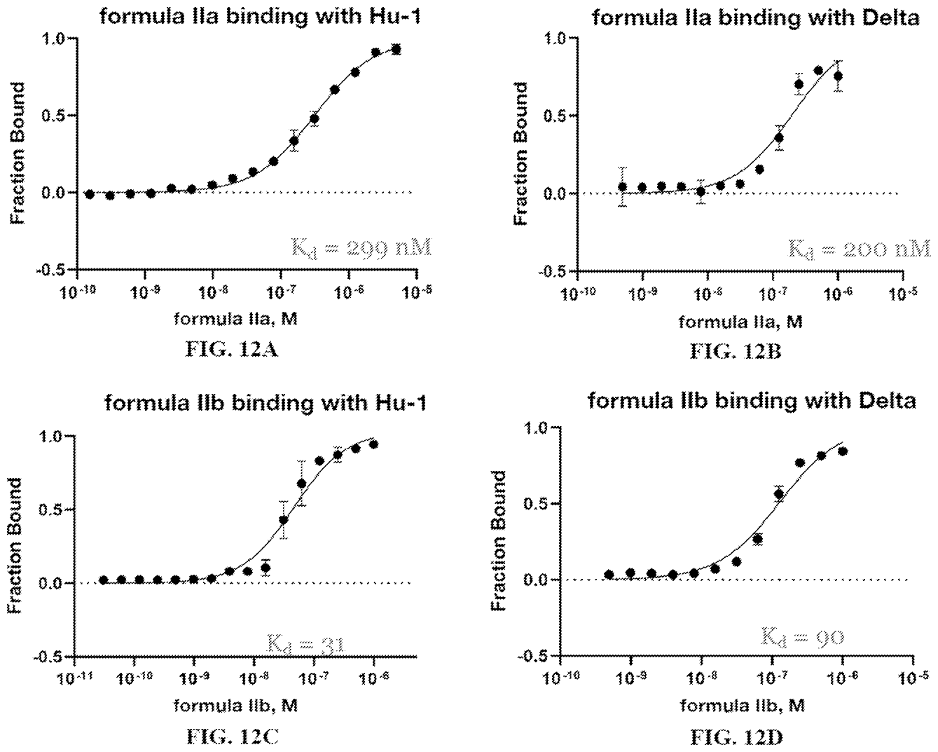

As shown in FIGS. 12A-D, in embodiments, the above-described MST methods are adaptable for use with variant protein complexes. For example, microscale thermophoresis (MST) may be used to analyze binding of formula (IIa) and formula (IIb) compounds with WT (“Hu-1”) and Delta S-RBD/ACE2 protein complexes. In another example, the MST assay with formula (IIa) and Hu-1 (FIG. 12A) provides a K_{d} of 299 nM. The MST assay with formula (IIa) and Delta (FIG. 12B) provides a K_{d} of 200 nM. The MST assay with formula (IIb) and Hu-1 (FIG. 12C) provides a K_{d} of 31 nM. The MST assay with formula (IIb) and Delta (FIG. 12D) provides a K_{d} of 90 nM. Thus, in embodiments, the binding affinity of compound formula (IIb) and/or pharmaceutical compositions comprising formula (IIb) with Hu-1 S-RBD/ACE complex is about 10-fold improved relative to Delta S-RBD/ACE complex.

### D. Measuring Cellular Toxicity of the Compounds

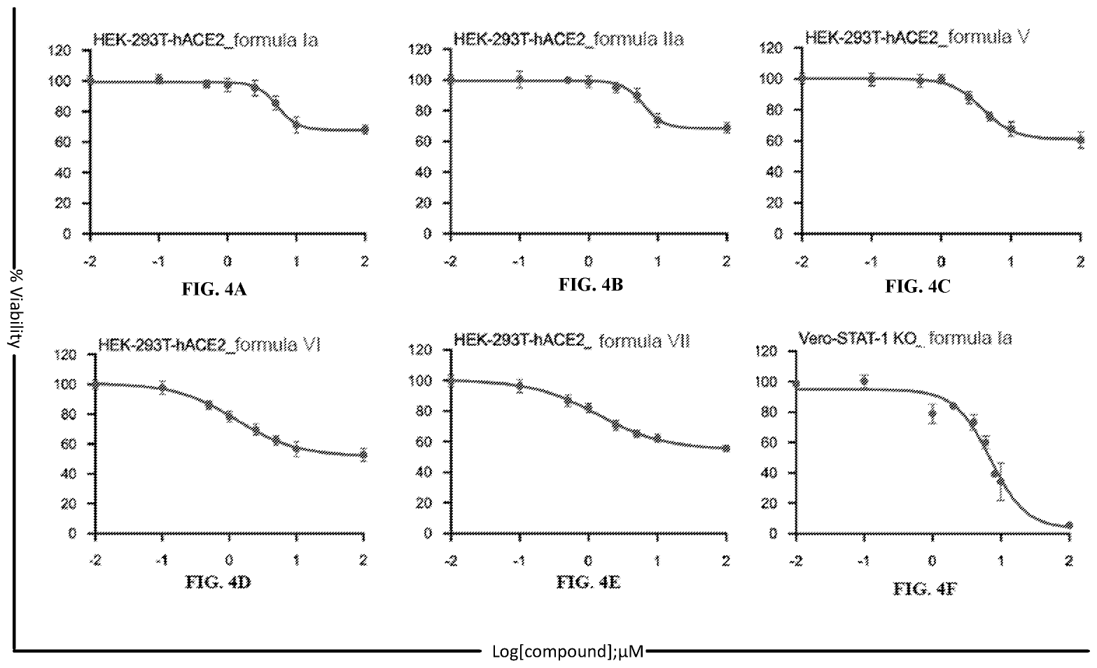

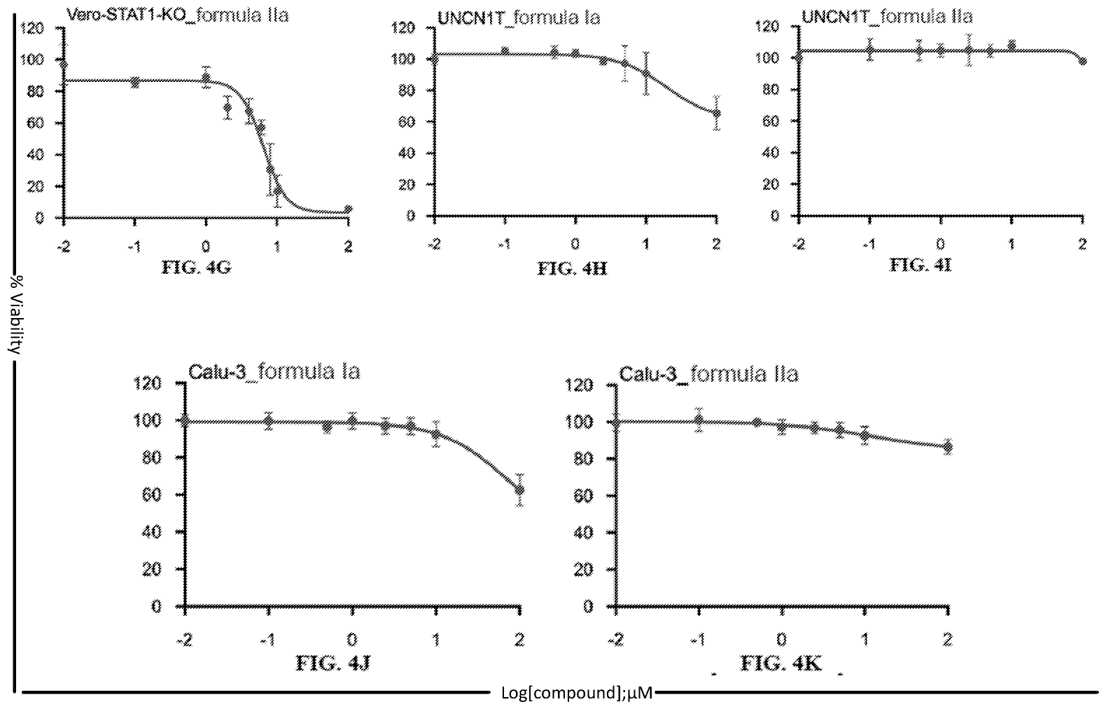

In some embodiments, the methods of the present invention provide for an accurate measure of cell cytotoxicity. Said methods are applied to candidate entry inhibitor compounds (e.g., the compounds of formula (Ia), formula (IIa), formula (V), formula (VI), and formula (VII), and the like). Assays were carried out in HEK293T-hACE2 cells (see FIGS. 4A-4K). First, cytotoxicity was computed in Vero-STAT1 knockout. UNCN1T, and Calu-3 cells for formula (Ia) (see FIG. 4A. FIG. 4F. FIG. 4H. FIG. 4J) and formula (IIa) (see FIG. 4B, FIG. 4G. FIG. 4I. FIG. 4K) using a colorimetric MTT assay that measures cell proliferation and viability. Next, the percent viability of the cells is plotted against the increasing concentration of the compounds, and the 50 percent cytotoxic concentration (CC₅₀) of each compound is computed using four-parameter variable slope sigmoidal dose-response models. In embodiments, in HEK293T-hACE2 cells, all five compounds exhibited CC₅₀ values above 100 μM (FIG. 4A-4E). In Vero-STAT1 knockout cells, formula (Ia) exhibited a CC₅₀ of 6.21 μM (FIG. 4F), and formula (IIa) provided a CC₅₀ of 7.13 μM (FIG. 4G) respectively. In other embodiments, in UNCN1T and Calu-3 cells formula (Ia) and formula (IIa) exhibited CC₅₀ values above 100 μM (FIGS. 4H-4K).

### E. Screening Entry Inhibition Potential

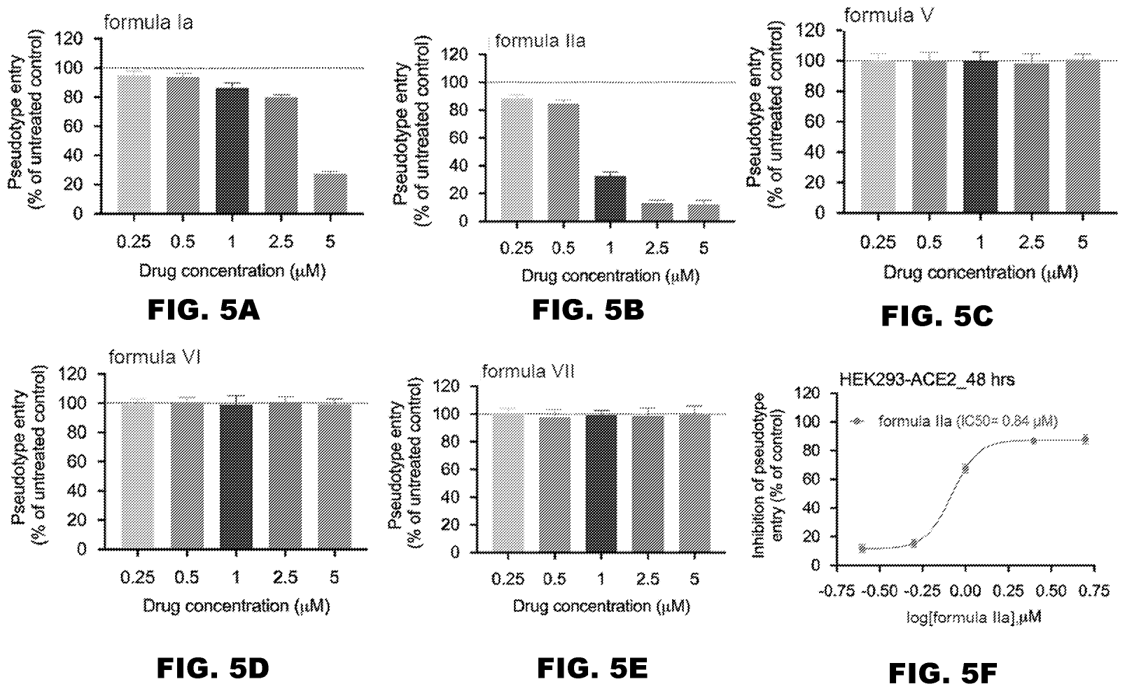

The disclosure provides a method of mimicking coronavirus host cell entry, wherein defective lentiviral particles expressing coronavirus Spike glycoprotein are replicated in first step. In said first step, lentiviral particles are adapted to incorporate surface-expressed SARS-CoV-2 Spike protein. Next, lentiviral particles are used to determine the relative efficiency of viral entry without an entry inhibition compound present, using human ACE2 expressing HEK-293T cells. Then. HEK-293T-hACE2 cells are treated with increasing concentrations of the compounds (0.25 to 5 μM) and thereafter transduced with pseudotyped lentiviral particles. Pseudotype viral entry is then calculated and measured relative to vehicle controls (DMSO) after 48 h. In embodiments, the compound of formula (Ia) displays a low level of viral entry inhibition with a slight increase at higher concentrations (FIG. 5A). In other embodiments, formula (IIa) treatment may result in significant and robust viral entry inhibition across a broad concentration range (FIG. 5B). In other embodiments, as measured relative to FIG. 5A and FIG. 5B, formula (V)(FIG. 5C), formula (VI)(FIG. 5D), and formula (VII)(FIG. 5E), may not prevent pseudovirus entry under the conditions of this in vitro experiment. The IC₅₀ value for formula (IIa) is 0.84 μM based upon a four-parameter variable slope sigmoidal dose-response model (FIG. 5F). In some embodiments, formula (IIa) exhibits a range of IC₅₀ values, including from 0.8-.0.85 μM or 0.9-1 μM. In other embodiments, formula (IIb) exhibits an even lower IC₅₀ value than formula (IIa), ranging from 0.1-0.2 μM. 0.22-0.3 μM. 0.32-0.6 μM, or 0.6-8 μM.

In embodiments, viral entry/replication may be blocked by the entry inhibitors by various means. For example, in one embodiment replication of SARS-COV-1 and/or SARS-COV-2 is inhibited by steric hindrance of bound small molecule compounds which prevent association of host ACE2 and S-RBD. In another example, viral entry/replication is inhibited by electrostatic repulsion of entry inhibitors and residues of RBD and/or the Spike protein. In other embodiments, a therapeutically effective amount of the herein disclosed entry inhibitors may block viral entry/replication by alternate means including covalent chemical interaction with the RBD/spike complex and/or side chains. In embodiments, the entry inhibitors may abstract protons, donate electrons, participate in Van Der Waals interactions, participate in quantum tunneling, and the like. Such interactions may disrupt ACE2 binding to Spike/RBD complex, therefore preventing entry and replication of the virus.

### F. Antiviral Efficacy Against Live SARS-COV-2 Isolates and Variants

In embodiments, the compounds of formula (Ia), formula (IIa), and formula (IIb) and pharmaceutical compositions of the same are shown to provide potent antiviral activity against SARS-COV-2. Methods of the present invention allow for measurement of antiviral efficacy for formula (Ia) and formula (IIa) against live SARS-COV-2 in UNCN1T and Vero-STAT1 knockout cells. UNCN1T is a human bronchial epithelial cell line and serves as a disease-relevant context for the target tissue of SARS-COV-2, lung epithelium. The Vero-STAT1 knockout cells are highly susceptible to viral infection due to the absence of STAT1, a transcription factor involved in the cellular antiviral interferon-mediated response. Therefore, in embodiments, these cells serve as a positive control for robust viral infection. In some embodiments, after using the cellular models described above. SARS-COV-2 replication kinetics are then measured at various time points (e.g., 24 and 48 hpi). Said measurements are determined in the presence of increasing concentrations of entry inhibitor compounds (e.g., formula (Ia), formula (IIa), formula (IIb), or any of the other entry inhibitor compounds described above such as formula (IIc), formula (IId), and formula (IIe).

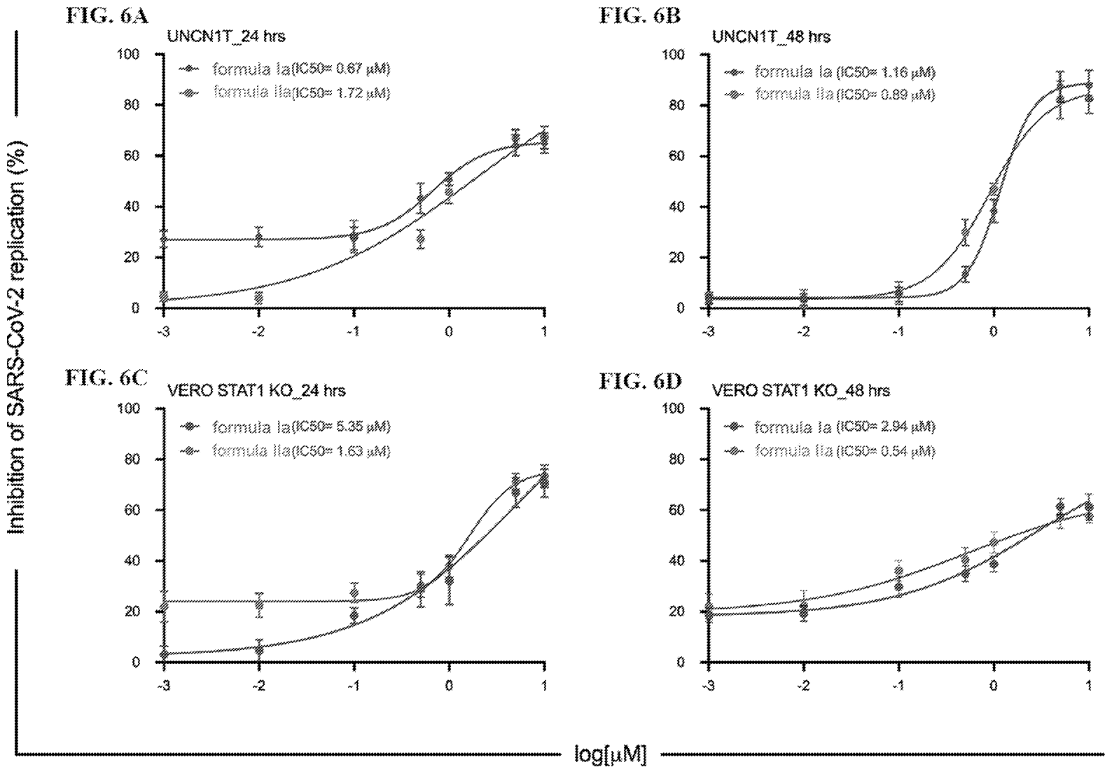

Applying the above-described method of the present invention, in one example formula (Ia) and formula (IIa) provided antiviral activity with IC₅₀ values of 0.67 and 1.72 μM, respectively, at 24 hpi (FIG. 6A). In another aspect, formula (Ia) and formula (IIa) have IC₅₀ values of 1.16 and 0.89 μM, respectively, at 48 hpi (FIG. 6B). In Vero-STAT1 knockout cells. 24 hpi formula (Ia) and 24hpi formula (IIa) have IC₅₀ values of 5.35 and 1.63 μM, respectively (FIG. 6C). In other embodiments, while at 48 hpi, the compounds of formula (Ia) and formula (IIa) have IC₅₀ values of 2.94 and 0.54 μM, respectively (FIG. 6D). In embodiments, formula (II)(e.g. formula (IIa)) exhibits a lower IC₅₀ value in FIGS. 6B-6D at all time points, consistent with its enhanced pharmacokinetic profile.

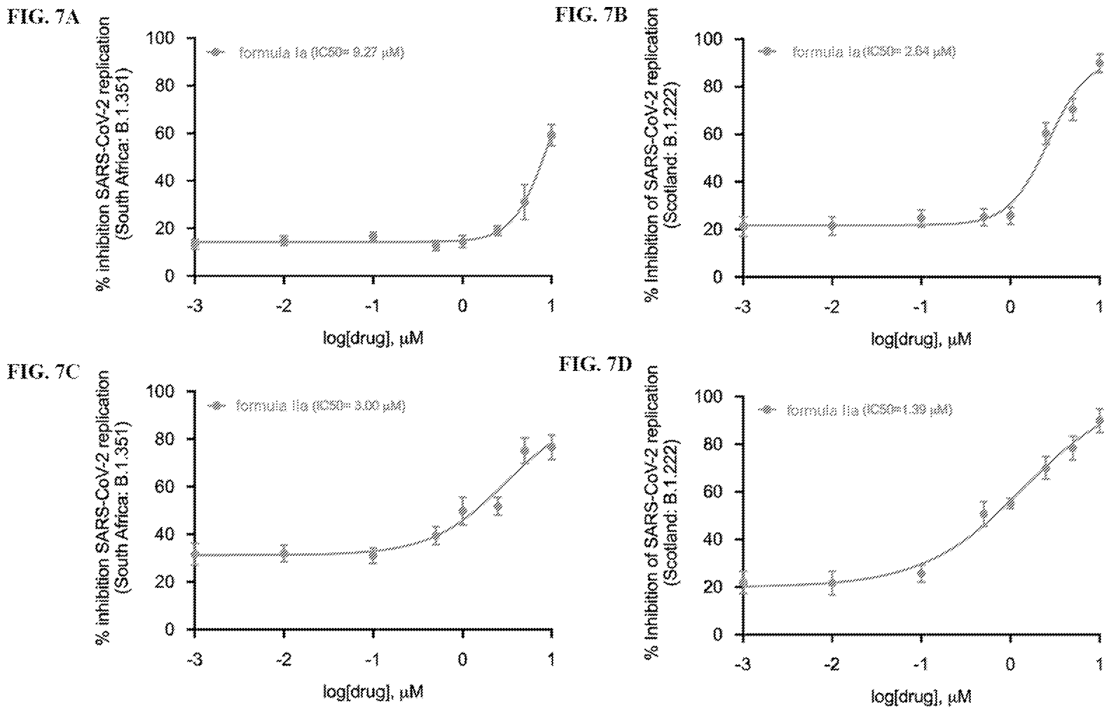

In embodiments, the antiviral efficacy of formula (Ia) and formula (IIa) may be further evaluated against variants of SARS-COV-2. As a proof of concept and based on their availability from BEI resources, the two mutant variants from South Africa (linage: B.1.351) and Scotland (linage: B.1.222) were selected. Based on SARS-COV-2 viral loads in the culture supernatant of Calu-3 cells, formula (Ia) and formula (IIa) showed comparable antiviral activity against the newly emerging variant strains compared to wild-type virus. In embodiments, formula (Ia) has an IC₅₀ value of 9.27 μM and 2.64 μM for South African (linage: B.1.351) variant (FIG. 7A) and Scotland (linage: B.1.222) variant (FIG. 7B) at 24 hpi, whereas formula (IIa) has an IC₅₀ value of 3.00 μM for South African (linage: B.1.351) variant (FIG. 7C) and 1.39 μM against Scotland (linage: B.1.222) variant at 24 hpi (FIG. 7D) respectively. The compound of formula (IIb) exhibits an even lower IC₅₀ value for the Scotland and South African variants. For example, in embodiments formula (IIb) provide a range of low IC₅₀ values comprising: 0.5-0.19 μM. 0.2-0.31 μM. 0.32-0.6 μM, or 0.6-8 μM.

Further to the above, said entry inhibitor methods and compositions are well adapted for use with a variety of SARS-COV-2 variants. In embodiments, said variants may include Variants Of Interest (VOIs) including and B.1.427 and B.1.429 (Feb. 26, 2021: Jun. 29, 2021). B.1.525 (Feb. 26, 2021). B.1.526 (Feb. 26, 2021). B.1.617.1 (May 7, 2021). B.1.617.3 (May 7, 2021), and P.2 (Feb. 26, 2021). In still other embodiments, said system for measuring antiviral efficacy against SARS-COV may be adapted for use with Variants Being Monitored (VBMs) including B.1.1.7 and Q lineages (Sep. 21, 2021). B.1.351 and descendent lineages (Sep. 21, 2021). P.1 and descendent lineages (Sep. 21, 2021). B.1.427 and B.1.429 (Sep. 21, 2021). B.1.427/B. 1.429 (Sep. 21, 2021). B. 1.525 (Sep. 21, 2021). B.1.526 (Sep. 21, 2021). B.1.617.1 (Sep. 21, 2021). B.1.617.3 (Sep. 21, 2021), and P.2 (Sep. 21, 2021). In other embodiments, it is contemplated that said compositions and methods are adapted for use with a variety of different coronaviruses, including MERS-COV, SARS-COV, NL63-COV, 229E-CoV, OC43-CoV, HKU1-CoV, WIV1-COV, MHV, HKU9-COV, PEDV-COV, or SDCV.

G. Antiviral Efficacy of Formula (Ia) and Formula (II) after Different Times of Addition to SARS-COV-2 Infected Cells

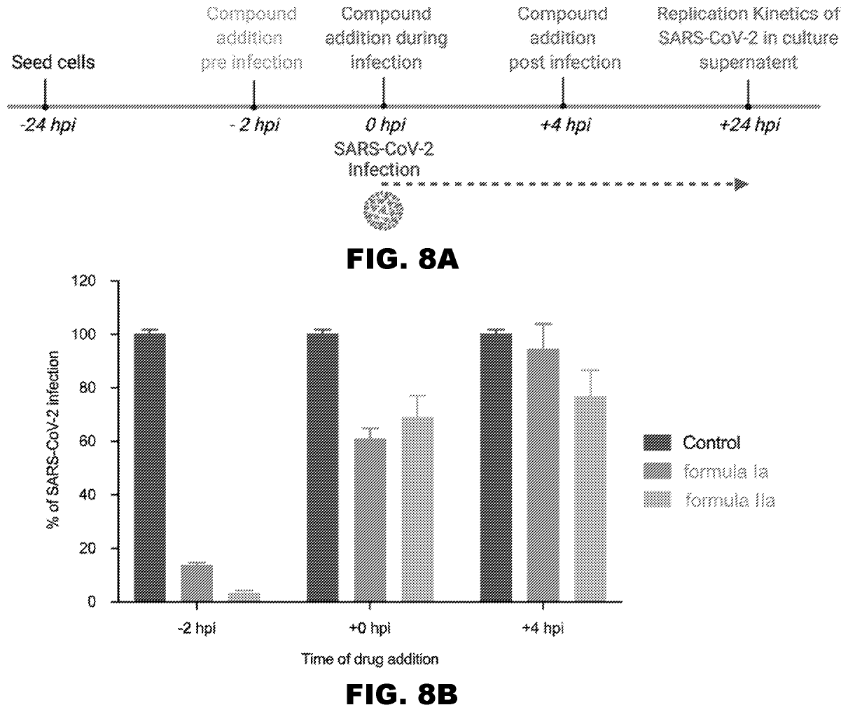

To determine at what stage of the viral life cycle the compounds of formula (Ia) and formula (IIa) impart their antiviral effect, the present methods include a time of addition assay. The experimental schema of the assay is described in FIG. 8A. In embodiments, it was observed that when both compounds were added −2 hpi, there was more than an 80% reduction in SARS-CoV-2 infectivity compared to vehicle controls. In some embodiments, when the compounds are added at the time of infection, a ˜40% reduction in SARS-COV-2 infectivity is observed. In other embodiments, and when the compounds are added +4 hpi, there is no significant difference in SARS-COV-2 infection compared with vehicle controls (FIG. 8B). In summary, in embodiments, formula (Ia) and formula (IIa) interact with the ACE2 and SARS-COV-2 Spike RBD binding interface, thereby preventing association of the protein complexes. In embodiments, when applied prior to infection, said compounds are more readily able to migrate to their targets to outcompete ACE2 and S-RBD/Spike Complex. Indeed, the present disclosure shows that the compounds of formula (IIb), when added −2 hpi, will exhibit a more than 85%. 90%, or 95% reduction in SARS-COV-2 infectivity compared to vehicle controls. Thus, in embodiments, formula (Ia) and formula (IIa) comprise pharmaceutical compositions that may be applied to a subject to both prevent and treat infection with SARS-COV-2.

### H. Compound Binding at ACE2:Spike RBD Induces Sidechain Conformational Changes in ACE2

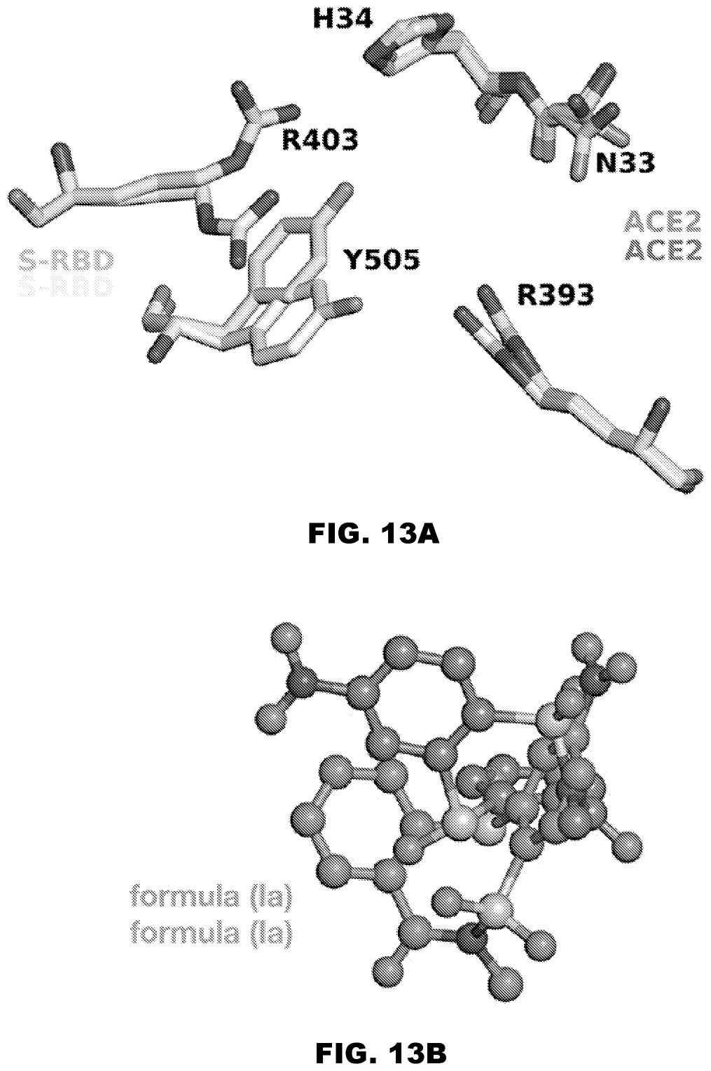

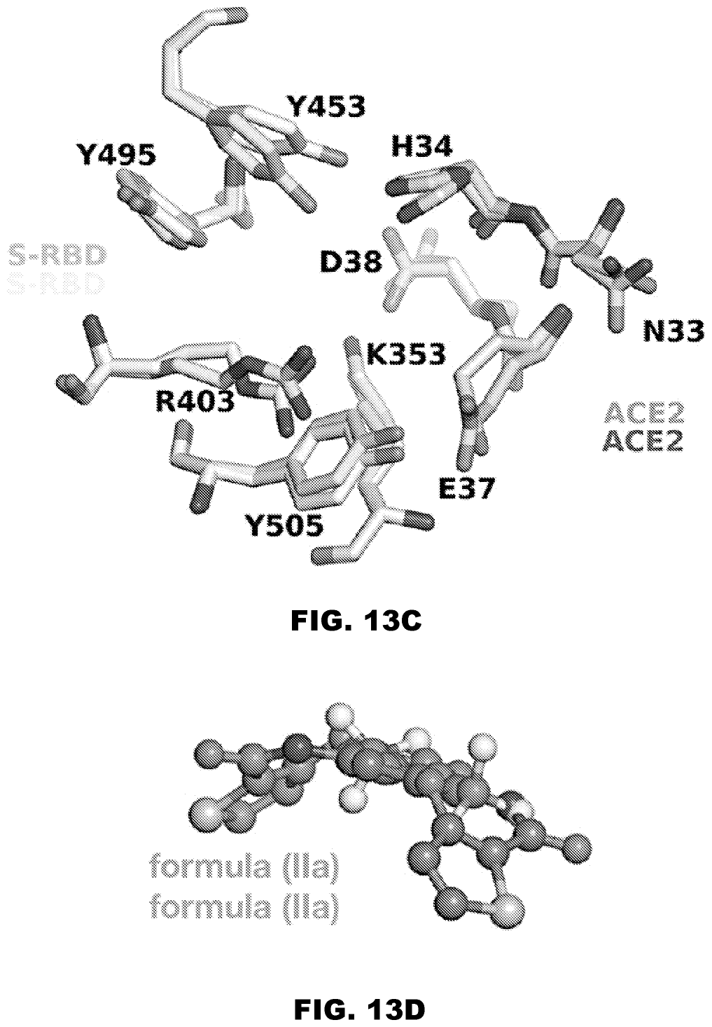

In embodiments, methods are disclosed to assess the impact of compound binding to the ACE2:Spike RBD complex. Specifically, induced-fit docking (IFD) is used to assess putative conformational changes induced by binding of the compounds. In embodiments, upon binding to ACE2:Spike RBD, the compounds of formula (Ia) and formula (II) induce significant sidechain conformational changes. In one embodiment, upon binding of the formula (Ia) to the S-RBD/ACE2 complex (as measured by flexible docking), residues Y505 and R403 are transposed in space (see FIG. 13A). In another embodiment, the sidechain conformation change of ACE2 residues N33, H34 and R393 are altered upon IFD (see modest rotation of N33, and significant distancing of Y505). In further embodiments, binding of the compound of formula (Ia) to the S-RBD/ACE2 complex significantly changes the mode of formula (Ia) binding to the binding pocket (FIG. 13B). Notably, in other embodiments, the sidechain conformation of several residues within the binding pocket remained unaltered. In embodiments, these residues include E406 and D405 of the S-RBD, and D30, E37. A386. E387. Q388, and P389 of ACE2.

In embodiments, significant sidechain conformational changes are observed upon IFD of formula (IIa) (FIG. 13C). In some embodiments, these residues include R403. Y453. Y495, and Y505 (S-RBD), and N33, H34, E37. D38, and K353 (ACE2). In other embodiments, sidechain conformation of S-RBD residues D405 and E406 and ACE2 residue R393 did not change upon IFD of formula (IIa). As seen with the compounds of formula (Ia), the binding conformation of formula (IIa) itself was also altered (FIG. 13D) upon binding to ACE2:Spike RBD. The present invention contemplates induction of sidechain conformational changes upon binding of formula (IIa) derivatives to ACE2:Spike RBD. Said derivatives comprise formula (IIb) (FIG. 2C), formula (IIc) (FIG. 2D), formula (IId) (FIG. 2E), and formula (IIe) (FIG. 2F). In other embodiments, the present invention contemplates induction of sidechain conformational changes upon binding to ACE2:Spike RBD of other herein disclosed compounds including formula (IIIa)(FIG. 2G), formula (IIIb)(FIG. 2H), formula (IIIc)(FIG. 2I), formula (IV)(FIG. 2J), formula (V)(FIG. 2K), formula (VI)(FIG. 2L), formula (VII)(FIG. 2M), formula (VIII)(FIG. 2N), formula (IX)(FIG. 2O), and formula (X)(FIG. 2P).

### I. Chemical Properties of Formula (IIa)

In embodiments, various biophysical properties of formula (IIa) are computed in addition to quantifying its synergistic effect with RDV. First. SwissADME web portal is used to compute various biophysical properties of all the compounds. Next, the kinetics of said compounds analyzed to show or refute a synergistic effect with RDV. In embodiments, said analysis of formula (II) (e.g. formula (IIa)) resulted in a Log Po/w value of at least 2.27, suggesting a high permeability and moderate solubility. Further, formula (IIa) has high gastrointestinal adsorption, but is not expected to inhibit CYP2C9. CYP2D6, and CYP3A4, suggesting low toxicity of the compound. In addition, in embodiments, formula (IIa) meets all of Lipnski's Rules of Five, and is predicted to exhibit high drug likeliness with no PAINS (Pan-assay interference compounds).

The use of combination therapies and repurposed drugs are also disclosed. Said therapies are adapted to target different stages of the viral life cycle, resulting in superior virological and physiological responses compared to monotherapy. This approach increases the overall efficacy of the treatment, reduces the dosage requirement of individual drugs, improves toxicity profiles, and lowers the chances of developing drug resistance. In other embodiments, in-vitro single-molecule protein folding experiments are used to design drugs targeting alternative folding conformation states of S-RBD and the like.

### J. Combinational Antiviral Efficacy of RDV/Formula (Ia) and RDV/Formula (IIa)

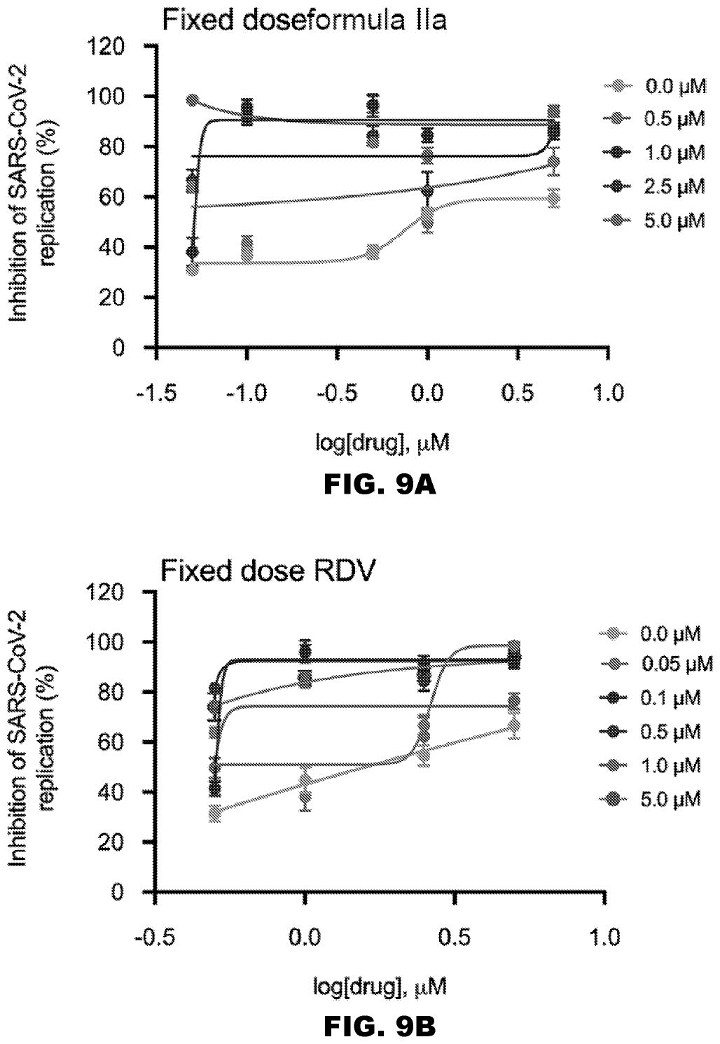

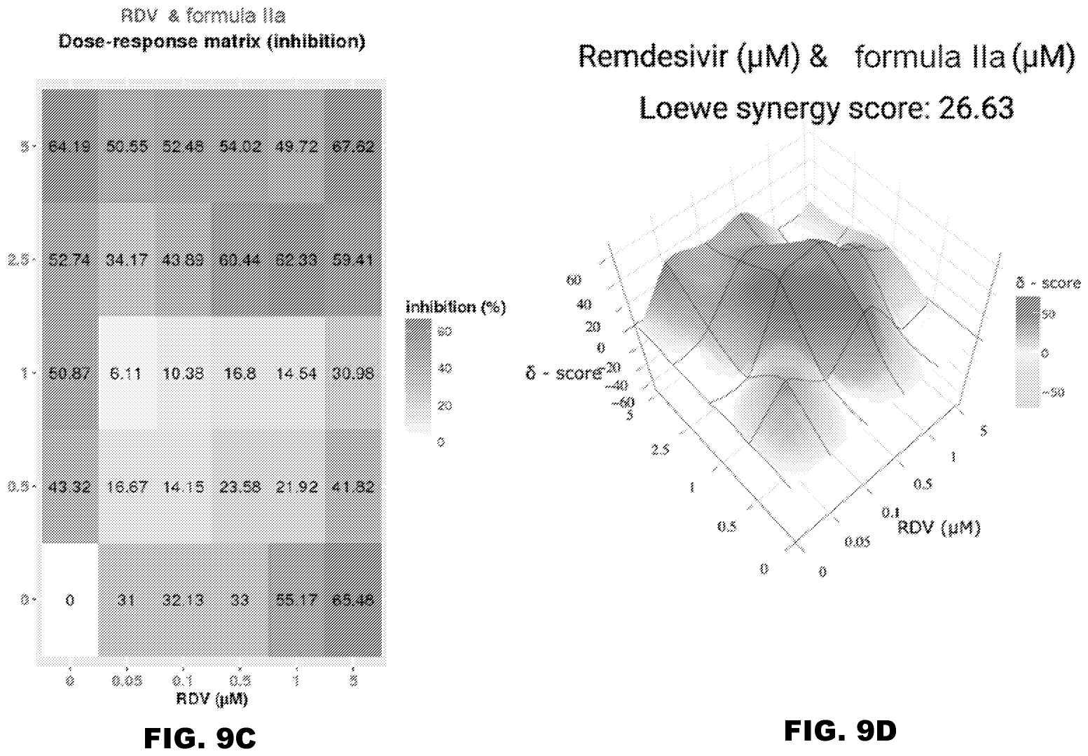

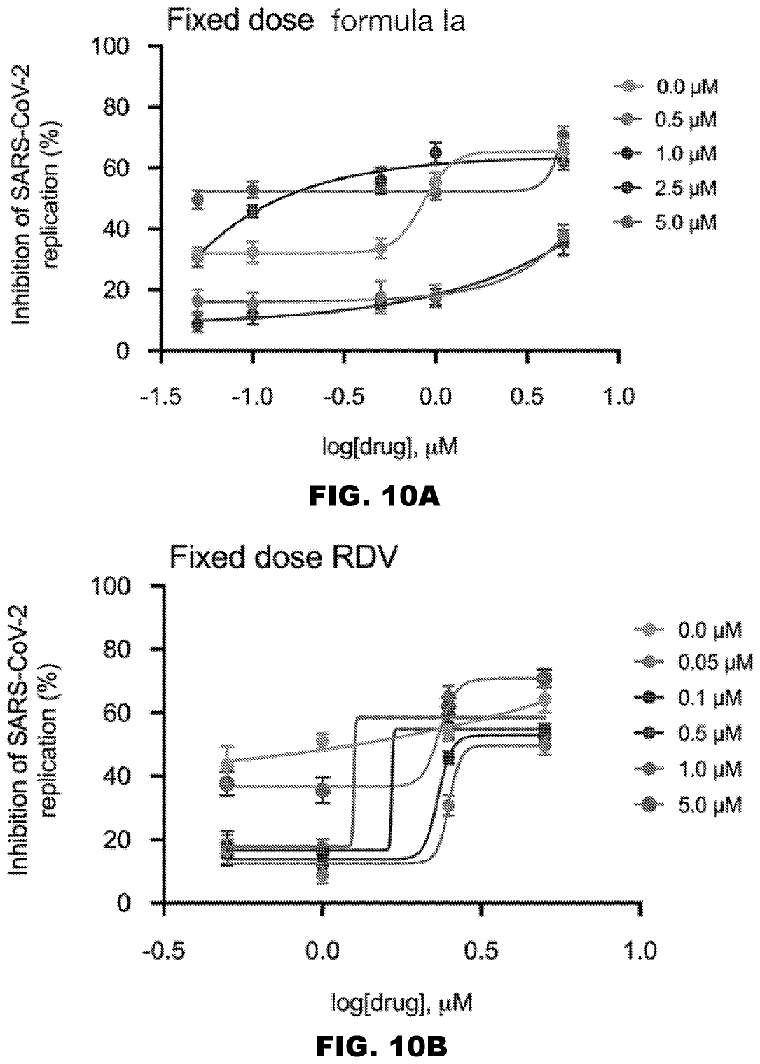

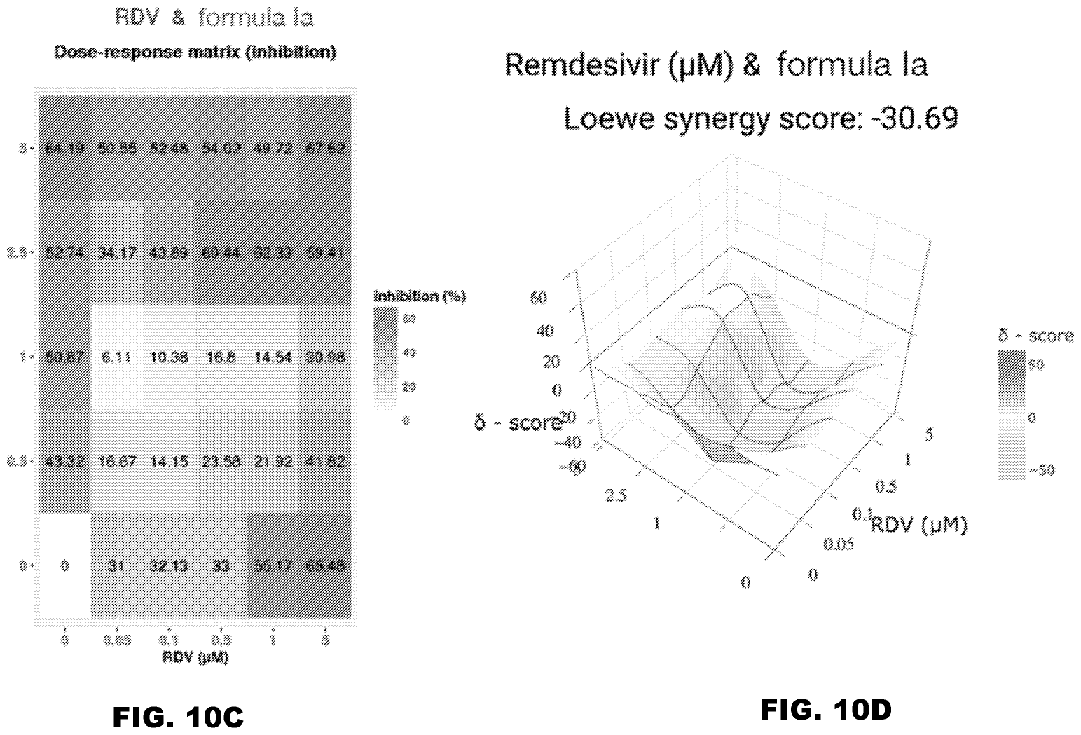

As described above, the effectiveness of formula (Ia) and formula (IIa) in blocking cellular entry of SARS-COV-2 was demonstrated. In addition, the disclosure provides a method to evaluate the combinational antiviral effect of formula (Ia)/formula (IIa) with RDV. As described above, the dose-response curve of RDV was determined at different fixed-dose combinations of formula (Ia) and formula (IIa) using SARS-COV-2 infected UNCN1T cells (see FIG. 9A and FIG. 10A). In some embodiments, the dose-response curves of formula (Ia) and formula (IIa) were determined at different fixed-dose combinations of RDV using SARS-COV-2 in infected UNCN1T cells (FIG. 9B and FIG. 10B). The dose-response percent inhibition matrix of single and combination treatment of RDV/formula (Ia) and RDV/formula (IIa) is shown in FIG. 9C and FIG. 10C, respectively. Finally, the 3-D interaction landscape of the combinational treatment was computed based on Loewe additive model using Synergy Finder v.2 in SARS-CoV-2 infected UNCN1T cells 24 hpi. Synergy maps highlight synergistic and antagonistic dose regions in red and green, respectively. A negative Loewe synergy score indicates an antagonistic drug combination, a score between 0) to 10 indicates the additive effect of drug combinations, and a score above 10 indicates a synergistic drug combination.

In embodiments. RDV/formula (Ia) combination has a Loewe synergy score of −30.69, indicating an antagonistic effect (FIG. 9D). In other embodiments, however. RDV/formula (IIa) has a Loewe synergy score of 26.64, indicating a synergistic effect (FIG. 10D). In embodiments, the synergistic interaction between RDV (an RdRp inhibitor) and formula (Ia) and/or formula (IIa) comprises at least a 28.3-fold and 2.3-fold reduction in dosages for formula (IIa) and RDV, respectively. In embodiments, these findings support the combinatorial use of the two compounds (RDV and formula (IIa)) against monotherapies. In embodiments, the combination therapy targeting two critical stages of the viral life cycle, namely, cell entry (formula (IIa)) and replication (RDV), enhances the overall therapeutic efficacy and reduces the chances of resistance associated with one drug. Notably studies with RDV and formula (Ia) resulted in some moderate aberrations in the data related to the Loewe synergy score. Notably, reduction in RDV dose is expected to reduce its adverse side effects that substantially limit its use in clinical settings. Thus, in the arena of antiviral therapeutics, the use of combination therapies with other potentially synergistic compounds (e.g., other nucleoside analogues, ribavirin, and the like) can be employed.

As described above, kinetic studies of the mechanism of S-RBD/Spike Complex inhibition revealed that formula (Ia) and formula (IIa) act as inhibitors (e.g. non-competitive inhibitors) of S-RBD/Spike Complex. In embodiments, as described above, it is shown that formula (IIa) does not binding individually to Hu-IS-RBD only and ACE2 only (see FIG. 11A and FIG. 11B). These results show that the entry inhibitors of the present invention specifically block S-RBD/Spike Complex through a novel and surprising mechanism. Further, in view of the specificity of the entry inhibitors for S-RBD/Spike Complex, the functions of cellular enzymes and other endogenous processes of the host mammal are not antagonized. In other embodiments, administration of formula I or formula (II) to a subject little to no humoral inflammatory immune response (e.g., low to no increase IFN-γ concentrations following entry inhibitor administration). In embodiments, said entry inhibitors block S-RBD/Spike Complex entry and replication by disruption of extracellular conformational changes normally observed in S-RBD/Spike and/or ACE2. In other embodiments, the entry inhibitors are valuable for diagnostics and/or to serve as a model for the study S-RBD/Spike Complex in the SARS-COV life cycle.

In embodiments, derivatives may be generated or synthesized by exchanging chemical groups of the entry inhibitors (e.g., formula (Ia), formula (IIa), formula (V), formula (VI), formula (VII), and the like) or precursors thereof. The derivatives may be subjected to various assays to determine the biological activity of replication inhibition in replication cell-based assays (e.g. lentiviral based pseudovirus assays, antiviral assays measuring IC₅₀, drug addition assays identifying stage of action of a derivative, and the like). In addition, modification to the entry inhibitors may be made to obtain derivatives with increased bioavailability, capability to cross membrane barriers, solubility, activity, or, e.g., stability. Derivatives may also be synthesized by, e.g., culturing a microorganism capable of producing an organic compound in a prescribed culture medium and reacting the organic compound obtained from the culture with the entry inhibitors or precursors thereof and with an additional reagent. Derivatives may also be synthesized by any organic chemical methodology. A compound library of chemical compounds containing derivatives of the entry inhibitors or a precursor thereof can be constructed. Such library enables random high-throughput screening of derivatives with improved characteristics, e.g., bioavailability, activity, stability, etc.

In particular embodiments, various derivatives for treatment of SARS-COV and other coronaviruses are employed.

In certain embodiments, the entry inhibitor compounds are derivatives of formula I, II, and/or III shown below:

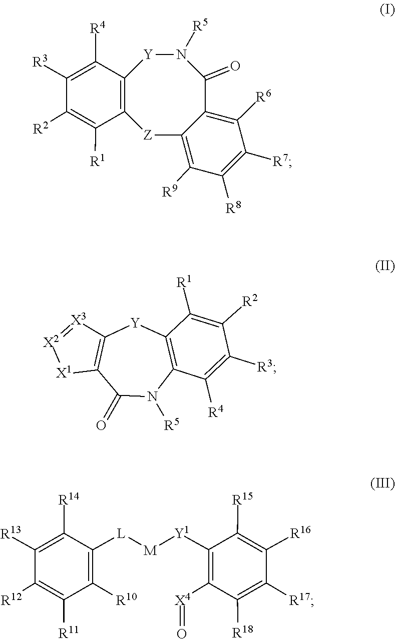

or
a salt thereof,
wherein each of R1, R2, R3, R4, R6, R7, R8 and R9 is independently hydrogen, halogen, nitro (—NO₂), aldehyde, carbonyl, carboxyl, hydroxyl, amine, aryl, heteroaryl, aryloxy, heteroaryloxy, —O(C₁-C₄)alkyl, —O(C₁-C₄)haloalkyl, (C₁-C₆)alkyl, or (C₁-C₆)alkyl substituted with one or more halogen;
R5 is independently hydrogen, halogen, aldehyde, carbonyl, carboxyl, hydroxyl, amine, aryl, heteroaryl, aryloxy, heteroaryloxy, (C₁-C₄)alkyl, (C₁-C₄)haloalkyl, —O(C₁-C₄)alkyl, or —O(C₁-C₄)haloalkyl;
each of R¹⁰. R¹¹. R¹², R¹³, R¹⁴, R¹⁵, R¹⁶, R¹⁷ and R¹⁸ is independently hydrogen, halogen, nitro (—NO₂), aldehyde, carbonyl, carboxyl, hydroxyl, amine, aryl, heteroaryl, aryloxy, heteroaryloxy, —O(C₁-C₄)alkyl, —O(C₁-C₄)haloalkyl, (C₁-C₆)alkyl, or (C₁-C₆)alkyl substituted with one or more halogen,
Y is O, S, S(═O), S(═O)₂, carbonyl, carboxyl, (C₁-C₆)alkyl, or (C₁-C₆)alkyl substituted with one or more halogen;
Z is O, S, S(═O), S(═O)₂, carbonyl, carboxyl, (C₁-C₆)alkyl, or (C₁-C₆)alkyl substituted with one or more halogen;
Y¹ is O, S, S(═O), S(═O)₂, nitro (—NO₂), aliphatic nitrile, carbonyl, carboxyl, (C₁-C₆)alkyl, or (C₁-C₆)alkyl substituted with one or more halogen;
X¹ is S, O, NH, or CR^{a1}, X² is N or CR^{a2}, X³ is N or CR^{a3}, and X⁴ is a (C₁-C₄)alkyl, wherein each of R^{a1}, R^{a2}, and R^{a3} is independently hydrogen, halogen, hydroxyl, (C₁-C₄)alkyl, (C₁-C₄)haloalkyl, aryl, heteroaryl, aryloxy, heteroaryloxy, —O(C₁-C₄)alkyl, or —O(C₁-C₄)haloalkyl;
L is absent or CR^{a4}, wherein R^{a4} is hydrogen, halogen, hydroxyl, (C₁-C₄)alkyl, (C₁-C₄)haloalkyl, aryl, heteroaryl, aryloxy, heteroaryloxy, —O(C₁-C₄)alkyl, or —O(C₁-C₄)haloalkyl; and
M is absent, NH, or N, wherein when M is N, M and X⁴ bind to form a cyclic group.

In some embodiments the formulae of I, II, or III can also be a derivative thereof or a pharmaceutically acceptable salt.

Any description of the formulas, including any R-group or chemical substituent, alone or in any combination, may be used in any chemical formula described herein, and formulae include all conformational and stereoisomers, including diastereomers, epimers, and enantiomers. The compounds described herein can have asymmetric centers. Accordingly the formulae containing an asymmetrically substituted atom may be isolated in optically active or racemic form. All chiral, diastereomeric, racemic forms and all geometric isomeric forms of a structure are intended, unless the specific stereochemistry or isomeric form is specifically indicated. Moreover, any feature of a composition disclosed herein may be used in combination with any other feature of a composition disclosed herein.

In other embodiments, the entry inhibitor compounds are derivatives of formula I, II, and/or III shown below:

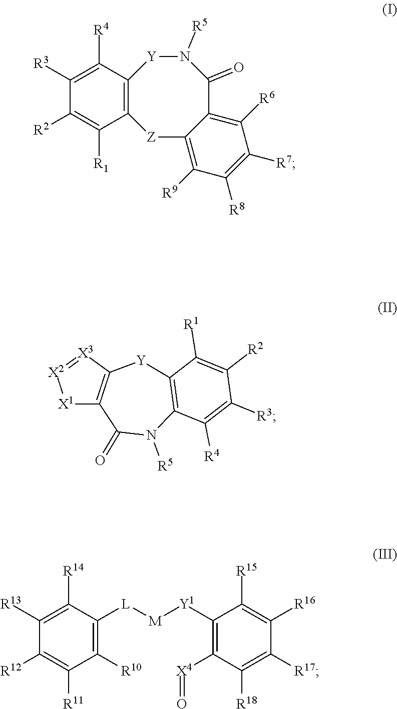

or
a salt thereof,
wherein each of R¹, R², R³, R⁴, R⁶, R⁷, R⁸ and R⁹ is independently hydrogen, nitro (—NO₂),
O(C₁-C₄)alkyl, —O(C₁-C₄)haloalkyl, (C₁-C₆)alkyl, or (C₁-C₆)alkyl substituted with one or more halogen;
R⁵ is independently hydrogen, halogen, (C₁-C₄)alkyl, (C₁-C₄)haloalkyl, —O(C₁-C₄)alkyl, or —O(C₁-C₄)haloalkyl;
each of R¹⁰, R¹¹, R¹², R¹³, R¹⁴, R¹⁵, R¹⁶, R¹⁷ and R¹⁸ is independently hydrogen, halogen, nitro (—NO₂), NH₂, O(C₁-C₄)alkyl, —O(C₁-C₄)haloalkyl, (C₁-C₆)alkyl, or (C₁-C₆)alkyl substituted with one or more halogen,
Y is O, S, S(═O), or S(═O)₂;
Z is O, S, S(═O), or S(═O)₂;
Y¹ is O, S, S(═O), S(═O)₂, nitro (—NO₂), or aliphatic nitrile;
X¹ is S, O, or CR^{a1}, X² is N or CR^{a2}, X³ is N or CR^{a3}, and X⁴ is a (C₁-C₄)alkyl, wherein each of R^{a1}, R^{a2}, and R^{a3} is independently hydrogen, halogen, (C₁-C₄)alkyl, (C₁-C₄)haloalkyl, —O(C₁-C₄)alkyl, or —O(C₁-C₄)haloalkyl;
L is absent or CR^{a4}, wherein R^{a4} is hydrogen, (C₁-C₄)alkyl, (C₁-C₄)haloalkyl, —O(C₁-C₄)alkyl, or —O(C₁-C₄)haloalkyl; and
M is absent, NH, or N, wherein when M is N, M and X⁴ bind to form a cyclic group.

The compounds or salts (including pharmaceutically acceptable salts) thereof binds to an interface of a SARS-COV-1 or SARS-COV-2 spike protein receptor binding domain (RBD) and a host cell ACE-2 receptor.

In embodiments, the compound has the following formula

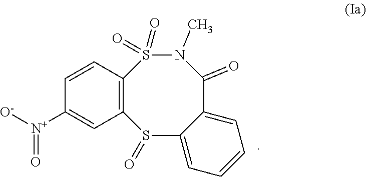 [SMILES: CN1C(=O)c2ccccc2S(=O)c2cc([N+](=O)[O-])ccc2S1(=O)=O]

In embodiments, the compound has one of the following formulae

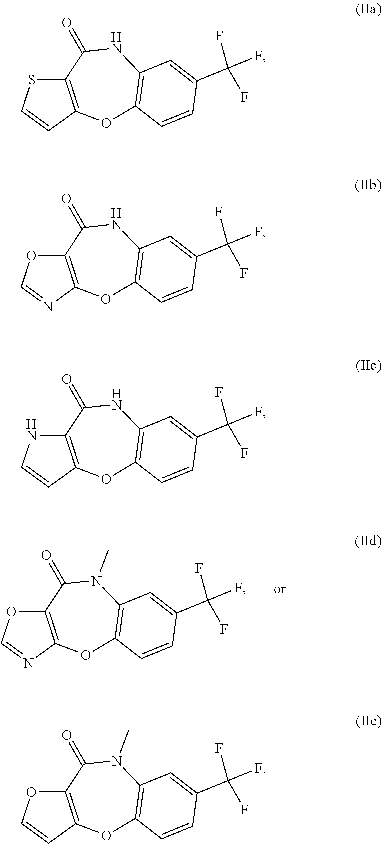

In embodiments, the compound or salt thereof has one of the following formulae

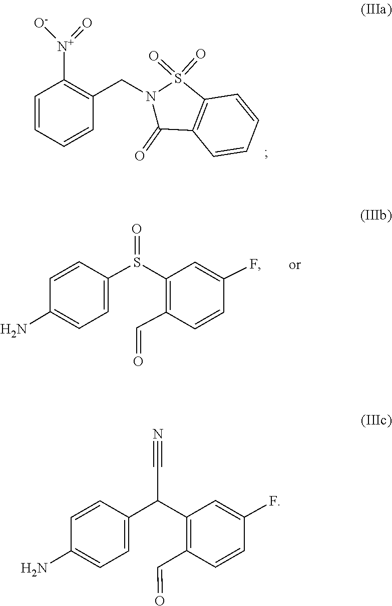 [SMILES: O=C1c2ccccc2S(=O)(=O)N1Cc1ccccc1[N+](=O)[O-] ; Nc1ccc(S(=O)c2cc(F)ccc2C=O)cc1 ; N#CC(c1ccc(N)cc1)c1cc(F)ccc1C=O]

In certain embodiments, the compounds of the present invention are suitable to inhibit virus replication, or treat or prevent a viral infection with a virus that uses ACE2 for entry that can be inhibited with the compounds of the present invention. In other embodiments, the compounds of the present invention are suitable to modulate the activity of mammalian (e.g., murine, human, aquatic mammal, and the like) ACE2 for any other ACE2-related indications known in the art. For example, the compounds of the present invention may disrupt or otherwise modulate the regulatory function of ACE2 in the renin-angiotensin system (RAS) responsible for regulation of cardiovascular and renal systems (e.g. blood-pressure regulation).

According to one embodiment of the invention, disclosed herein is a method of inhibiting the replication of a virus comprising administering a compound with the core formula I, II, or III:

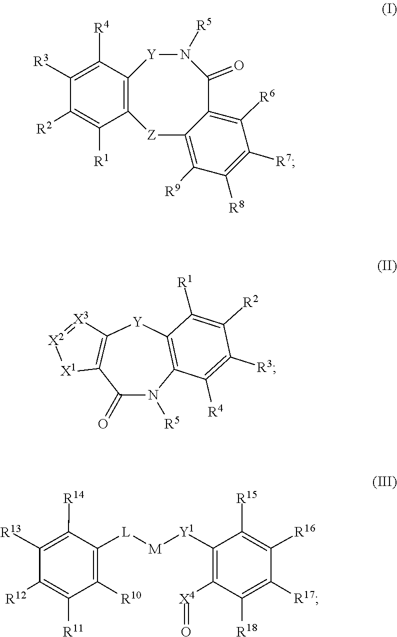

or
a salt thereof,
wherein each of R¹, R², R³, R⁴, R⁶, R⁷, R⁸ and R⁹ is independently hydrogen, halogen, nitro (—NO₂), aldehyde, carbonyl, carboxyl, hydroxyl, amine, aryl, heteroaryl, aryloxy, heteroaryloxy, —O(C₁-C₄)alkyl, —O(C₁-C₄)haloalkyl, (C₁-C₆)alkyl, or (C₁-C₆)alkyl substituted with one or more halogen;
R⁵ is independently hydrogen, halogen, aldehyde, carbonyl, carboxyl, hydroxyl, amine, aryl, heteroaryl, aryloxy, heteroaryloxy, (C₁-C₄)alkyl, (C₁-C₄)haloalkyl, —O(C₁-C₄)alkyl, or —O(C₁-C₄)haloalkyl;
each of R¹⁰, R¹¹, R¹², R¹³, R¹⁴, R¹⁵, R¹⁶, R¹⁷ and R¹⁸ is independently hydrogen, halogen, nitro (—NO₂), aldehyde, carbonyl, carboxyl, hydroxyl, amine, aryl, heteroaryl, aryloxy, heteroaryloxy, —O(C₁-C₄)alkyl, —O(C₁-C₄)haloalkyl, (C₁-C₆)alkyl, or (C₁-C₆)alkyl substituted with one or more halogen,
Y is O, S, S(═O), S(═O)₂, carbonyl, carboxyl, (C₁-C₆)alkyl, or (C₁-C₆)alkyl substituted with one or more halogen;
Z is O, S, S(═O), S(═O)₂, carbonyl, carboxyl, (C₁-C₆)alkyl, or (C₁-C₆)alkyl substituted with one or more halogen;
Y¹ is O, S, S(═O), S(═O)₂, nitro (—NO₂), aliphatic nitrile, carbonyl, carboxyl, (C₁-C₆)alkyl, or (C₁-C₆)alkyl substituted with one or more halogen;
X¹ is S, O, NH, or CR^{a1}, X² is N or CR^{a2}, X³ is N or CR^{a3}, and X⁴ is a (C₁-C₄)alkyl, wherein each of R^{a1}, R^{a2}, and R^{a3} is independently hydrogen, halogen, hydroxyl, (C₁-C₄)alkyl, (C₁-C₄)haloalkyl, aryl, heteroaryl, aryloxy, heteroaryloxy, —O(C₁-C₄)alkyl, or —O(C₁-C₄)haloalkyl;
L is absent or CR^{a4}, wherein R^{a4} is hydrogen, halogen, hydroxyl, (C₁-C₄)alkyl. (C₁-C₄)haloalkyl, aryl, heteroaryl, aryloxy, heteroaryloxy, —O(C₁-C₄)alkyl, or —O(C₁-C₄)haloalkyl; and
M is absent, NH, or N, wherein when M is N, M and X⁴ bind to form a cyclic group.

Any description of the formulas, including any R-group or chemical substituent, alone or in any combination, may be used in any chemical formula described herein, and formulae include all conformational and stereoisomers, including diastereomers, epimers, and enantiomers. The compounds described herein can have asymmetric centers. Accordingly the formulae containing an asymmetrically substituted atom may be isolated in optically active or racemic form. All chiral, diastereomeric, racemic forms and all geometric isomeric forms of a structure are intended, unless the specific stereochemistry or isomeric form is specifically indicated. Moreover any feature of a composition disclosed herein may be used in combination with any other feature of a composition disclosed herein. In embodiments, formula (Ia) comprises: the entry inhibitor category I, comprising formula (Ia)(IC₅₀=<0.25 μM): 6-methyl-2-nitrodibenzo[d,g][1.6.2]dithiazocin-7(6H)-one 5,5, 12-trioxide.

In certain embodiments, the compounds and methods are designed for use as entry inhibitors of Nidovirales viruses, such as Coronaviridae viruses. SARS viruses, and the like. In other embodiments the entry inhibitors act against other RNA viruses, including Ebola, influenza. MERS-COV and Venezuelan equine encephalitis virus.

In certain embodiments, the present invention also includes pharmaceutical compositions comprising the herein disclosed entry inhibitors (see Table 2) or a pharmaceutically acceptable salts thereof, and/or and a pharmaceutically acceptable carrier.

If pharmaceutically acceptable salts of the compounds of this invention are utilized in these compositions, those salts may be derived from inorganic or organic acids and bases. Included (as an exemplary listing) among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide. 2-hydroxyethanesulfonate, lactate, maleate, methanesulfonate. 2-naphthalenesulfonate, nicotinate, oxalate, pamoate, pectinate, persulfate. 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts. N-methyl-D-glucamine, and salts with amino acids such as arginine, lysine, and so forth.

In certain embodiments, the compounds utilized in the compositions and methods of this invention may also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion. The antiviral compositions provided herein may optionally include one or more additional components, such as carriers, stabilizers, immune system stimulating materials, disinfectants, chemically or otherwise inactivated viral material, or additional viral inhibitory compounds. In certain embodiments, pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

It should also be understood that a specific dosage and treatment regimen for any particular patient will depend upon a variety of factors, including the activity of the pharmaceutical composition, the age, body weight, general health, sex, diet, time of administration, rate of excretion, drug combination, and the judgment of the treating physician and the severity of the particular disease being treated. The amount of active ingredients will also depend upon the particular compound and/or anti-viral agent, if present, in the composition.

The dosage ranges for administration of the entry inhibitors or derivatives thereof to a subject, are those which produce the desired affect whereby symptoms of infection are ameliorated. In particular, the compounds of the present invention are effective against Nidovirales viruses such as SARS-COV-2 that comprise a S-RBD domain and form an S-RBD/ACE2 complex during the life cycle of infection. For example, as used herein, a pharmaceutically effective amount for a SARS-COV-1 and SARS-COV-2 infection refers to the amount administered so as to maintain an amount which suppresses or inhibits circulating virus throughout the period during which infection is evidenced such as by the presence of anti-viral antibodies, presence of culturable virus, and/or the presence of viral antigen in patient sera, or symptoms that are identifiable by a medical professional. For example, the presence of anti-viral antibodies can be determined through use of standard ELISA or Western blot assays.

Dosages generally vary with age, extent of the infection, body weight, immune tolerance, and counterindications, if any. The dosage will also be determined by the existence of any adverse side effects that may accompany the compounds. It is desirable, whenever possible, to keep adverse side effects to a minimum. One skilled in the art can easily determine the appropriate dosage, schedule, and method of administration for the formulation of the composition being used in order to achieve the desired effective concentration in the individual patient. However, the dosage may vary, for example, from between about 0.001 mg/kg/day to about 150 mg/kg/day, or optionally between about 1 to about 50 mg/kg/day.

In one embodiment, an ACE2: SARS-COV-2 Spike RBD entry inhibitor compound, composition, or pharmaceutical composition (“entry inhibitor”), or combinations of said entry inhibitors, are administered to a subject at a concentration of between 0.1 mg/ml and about any one of 0.5, 1, 5, 10, 15 20, 25, 30, 35, 40, 45, 50, 55, 60, 65, 70, 75, 80, 85, 90, 95, 100, 110, 120, 130, 140, 150, 160, 170, 180, 190, or 200 mg/ml. +/−10% error.

In another embodiment, the entry inhibitor compound or a pharmaceutically acceptable salt thereof are administered to a subject at a dose of between about 0.01 and 100.0 or 200.0 mg/kg of body weight of the recipient subject. In certain embodiments, depending on the type and severity of the SARS-COV-S-related disease, about 1 μg/kg to 50 mg/kg (e.g., 0.1-20 mg/kg) of compound is an initial candidate dosage for administration to the patient, whether, for example, by one or more separate administrations, or by continuous infusion. In another embodiment, about 1 μg/kg to 15 mg/kg (e.g., 0.1 mg/kg-10 mg/kg) of compound is an initial candidate dosage for administration to the patient. A typical daily dosage might range from about 1 μg/kg to 100 mg/kg or more, depending on several factors, e.g., the particular mammal being treated, the clinical condition of the individual patient, the cause of the disorder, the site of delivery of the agent, the method of administration, the scheduling of administration, and other factors known to medical practitioners. However, other dosage regimens may be useful.

The antiviral compositions of the invention may be administered to the receiving subject in any medically effective manner, including enteral, parenteral, topical, transmucosal, intramuscular, intravenous, and inhalation delivery methods.

In certain embodiment, the compositions of this invention are formulated for pharmaceutical administration to an organism such as a mammal, or human being. In some embodiments, the compositions of this invention are formulated for pharmaceutical administration to livestock, domesticated animals, wild animals (e.g., vector animals such as bats, pangolins, and the like), and/or aquatic mammals. Such pharmaceutical compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

In certain embodiments, the compositions are administered orally, intraperitoneally or intravenously. The compound or pharmaceutical compositions may also be administered by a non-oral route (e.g., ophthalmic, inhalation and transdermal). Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a nontoxic parenterally-acceptable diluent or solvent, for example as a solution in 1,3-butanediol. Among the acceptable vehicles and solvents that may be employed are water. Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as those described in Pharmacopeia Helvetica or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added. In certain embodiments, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

In embodiments, administered parenterally, the pharmaceutical composition may be formulated in a unit dosage injectable form (e.g., solution, suspension, emulsion) with at least one pharmaceutically acceptable excipient. Such excipients are typically nontoxic and non-therapeutic. Examples of such excipients are water, aqueous vehicles such as saline. Ringer's solution, dextrose solution, and Hank's solution and non-aqueous vehicles such as fixed oils (e.g., corn, cottonseed, peanut and sesame), ethyl oleate, and isopropyl myristate. Sterile saline is a preferred excipient. The excipient may contain minor amounts of additives such as substances that enhance solubility, isotonicity, and chemical stability, e.g., antioxidants, buffers, and preservatives. When administered orally (or rectally) the compounds will usually be formulated into a unit dosage form such as a table, capsule, suppository, or cachet. Such formulations typically include a solid, semi-solid or liquid carrier or diluent. Exemplary diluents and excipients are lactose, dextrose, sucrose, sorbitol, mannitol, starches, gum acacia, calcium phosphate, mineral oil, cocoa butter, oil of theobroma, alginates, tragacanth, gelatin, methylcellulose, polyoxyethylene, sorbitan monolaurate, methyl hydroxy benzoate, propyl hydroxybenzoate, talc and magnesium stearate.

In some embodiments, the pharmaceutical composition according to the invention is administered intravenously. In certain embodiments, the pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. In certain embodiments, topical application for the lower intestinal tract can be affected in a rectal suppository formulation or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol. 2-octyldodecanol, benzyl alcohol and water. For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

In embodiments, the pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. In embodiments, the amount of entry inhibitor compound present in the above-described composition should be sufficient to cause a detectable decrease in a disease state in a subject and/or a measurable decrease in viral replication.

In certain embodiments, the invention provides for methods of treating an organism suspected of having a SARS-COV-1 and/or SARS-COV-2 infection. In certain aspects such method may include steps for identifying organisms suspected of having a SARS-COV-1 and/or SARS-COV-2 infection. Such identification can be conducted by diagnostic procedures specific for the particular viral infection. This may include detecting symptoms of the virus infection, and detecting virus-specific antigens, antibodies, or nucleic acids in a biological sample. The term “biological sample.” as used herein may include cell cultures or extracts thereof: biopsied material obtained from a mammal or extracts thereof; and blood, saliva, urine, feces, tears, or other body fluids or extracts thereof. The term “biological sample” also includes living organisms.

In certain embodiments, the invention provides methods for administering the pharmaceutical composition of the present invention to an organism that is suspected to have been exposed or will be exposed to SARS-COV-1 and/or SARS-COV-2. In certain embodiments, the components or pharmaceutical compositions of the present invention provide prophylactic and/or therapeutic effects nearly immediately upon administration.

### EXAMPLES

The following examples are not intended to limit the scope of the claims.

### Example 1—Identification of Potential Inhibitor Using Computer-Aided Drug Design

In embodiments, the ˜8 million drug-like compounds were run through an in silico docking program to identify potential SARS-COV-2 entry inhibitors (see FIG. 1A). The library consists of compounds from May Bridge Hitfinder compounds, small molecules from the Zinc database (zinc.docking.org), ChEMBL, Bingo, JChemforExcel, ChemDiff, and BindingMOAD (https://www.click2drug.org/index.php#Databases), and all compounds were prepared in their docking-ready conformation using ‘LigPrep’ program of Schrödinger Suite (Schrödinger LLC. NY). The S-RBD/ACE2 complex [Protein Databank Entry 6M0J: Lan et al. 2020] was used for in silico screening. A docking-ready structure was generated by the “Protein Preparation Wizard” of the Schrödinger Suite (Schrödinger LLC. NY), which adds the hydrogen atoms, missing sidechains, and assigns protonation states to histidine, glutamine, and asparagine residues together with the optimization of the hydrogen atoms' orientation. The resulting structure was energy minimized using OPLS_2005 forcefield for 10.000 iterations to remove steric conflicts. Potential compound binding sites were identified by SiteMap (Schrödinger Suite) and SiteID (SybylX-2.1, Certera, Princeton, NJ). A binding pocket present at the interface of S-RBD/ACE2 was selected for the docking of the library compounds (details of interface residues and pocket are given in Results' section. The Glide program of Schrödinger Suite with SP (Simple Precision) was used in initial docking in a grid box of 20×20×20 Å³ size. The top 500 compounds based on the docking score were re-docked using the XP (Extra Precision) option of Glide. These results were then manually visualized for the interactions between compounds and protein (S-RBD and ACE2). Five compounds (formulas (I). (II). (IIIa). (IIIb), and (IIIc)) were finally selected for testing their in vitro inhibitory activity in cell-based assays (Table 2). To gain insight into the binding mode of these compounds and accessing the flexibility of the binding site residues, flexible docking using the IFD program of the Schrödinger Suite is carried out.

Regarding molecular modeling of S-RBD of B.1.351 variant in complex with ACE2, the S-RBD/ACE2 complex crystal structure of the P.1 variant (PDB entry 7NXC) was utilized. Mutation K417T in the P.1 variant was altered to K471N to generate the structure of the B. 1.351 S-RBD variant using the Prime modeling program of Schrodinger Suite. This structure was then used in ‘Induced Fit Docking’ of formula (Ia) and formula (IIa).

### Example 2—Reagents and Cell Lines

RDV (GS-5734) was obtained from Selleck Chemicals LLC (Houston, TX). The SARS-CoV-2 entry inhibitors were obtained from MolPort (Riga, Latvia). Calu-3 (ATCC HTB-55). Vero E6 (CRL-1586), and Vero-STAT1 knockout cells (CCL-81-VHG) were obtained from ATCC. Vero E6 and Vero-STAT1 knockout cells were cultured in DMEM containing 10% fetal bovine serum (FBS). 2 mM 1-glutamine. 100 units/ml penicillin. 100 units/ml streptomycin, and 10 mM HEPES (pH 7.4). Calu-3 cells were cultured in Eagle's Minimum Essential Medium (ATCC 30-2003) containing 10% FBS. UNCN1T cells (a human bronchial epithelial cell line; Kerafast catalog number ENC011) were cultured in BEGM media (Bronchial Epithelial Cell Growth Medium: Lonza catalog number CC-3170) in FNC (Athena Enzyme Systems catalog number 0407) coated 96-well plates. All other reagents (molecular biology grad fine chemicals) used in the study were purchased from Sigma-Aldrich (St. Louis, MO) unless otherwise mentioned.

### Example 3—ACE2: SARS-COV-2 Spike RBD Inhibitor Screening Assay

Six compounds ((formulas (I). (II). (IIIa). (IIIb). (IIIc), and (IV)) were tested in triplicate in the concentration range from 0.25 to 5.00 μM using the ACE2: SARS-COV-2 Spike Inhibitor Screening assay kit (BPS Bioscience catalog number 79936) per the manufacturer's instruction. In brief, the ACE2 protein was thawed on ice, diluted to 1 μg/ml using IX PBS, applied at 50 μl/well to the 96-well nickel-coated plate provided in the kit, and incubated for 1 h at room temperature with gentle shaking. The plate was then washed three times with 1× immune buffer 1, 100 μl of 1× blocking buffer 2 was added/well and incubated at room temperature for 10 min with slow shaking. Next. 10 μl of the compounds was added in triplicate and incubated for 1 h at room temperature with slow shaking. Ten μl of 5% DMSO was used as vehicle control. After that. 5 nM SARS-COV-2 Spike (RBD)-Fc (20 μl) was added/well and incubated at room temperature for 10 min with slow shaking. The plate was rewashed three times with 1× immune buffer 1, 100 μl of 1× blocking buffer 2 was added/well and incubated at room temperature for 10 min with slow shaking. Next. 1:1000 diluted anti-mouse-Fc-HRP was added/well of the plate and incubated for 1 h at room temperature with slow shaking. Finally, the plate was rewashed as described above and HRP substrate was added to produce chemiluminescence, which was measured using a SpectraMax i3× multi-mode plate reader (Molecular Devices. San Jose, CA).

### Example 4—MTT Cell Viability Assay

The MTT cell viability assay of the preferred embodiment utilized HEK-293T-hACE2. UNCN1T. Vero-STAT1 knockout, and Calu-3 cells seeded at the density of 20,000 cells/well in a 96-well plate containing 100 μl complete media specific for each cell type. Cells were incubated for 12 h at 37° C., in a humidified 5% CO₂ incubator for adherence. After 12-h incubation, the media was replaced with fresh media, and HEK-293T-hACE2 cells were treated with the five compounds at concentrations ranging between 0.001 to 100 μM. The Calu-3. UNCN1T, and Vero STAT1 knockout cells were treated with formula (Ia) and formula (IIa). Untreated cells were considered a negative control, and DMSO treated cells were considered vehicle controls. After the treatment, cells were incubated at 37° C., in humidified 5% CO₂ incubator. Seventy-two hour posttreatment. 20 μl of MTT substrate (5 mg/ml) was added to each well and incubated for 4 additional hours at 37° C., in the dark. Then the culture media was carefully removed, and blue formazan crystals were dissolved in 200 μl of DMSO, and the purple color was read at 595 nm with a reference filter of 620 nm.

In alternate embodiments. Vero and HepG2 cells were seeded at the density of 15,000-25.000 cells/well in a 96 well plate containing 100 μL of complete DMEM (Gibco. USA) supplemented with 10% FBS (Gibco. USA) and 1% Penstrep (Gibco. USA). Cells were incubated for 12 hours at 37° C., in humidified 5% CO₂ incubator for the adherence. After 12 h incubation, the media was replaced with fresh media and cells were treated with the compound concentrations ranging between (XX to YY). Untreated cells were considered as negative control and DMSO treated cells were considered as vehicle. After the treatment, cells were again incubated at 37º C in a CO₂ incubator. 48-hours post treatment. 20 μL of MTT substrate (5 mg/mL) was added in each well and incubated for 4 hours at 37ºC in dark. 4 hours post incubation, media was removed carefully and blue formazan crystals were dissolved in 200 μL of DMSO and the purple color was read at 595 nm.

### Example 5—Production and Titration of Lentiviral-Based Pseudovirus Expressing SARS-CoV-2 Spike Glycoprotein

For pseudotyping (FIGS. 5A-5F), lentiviral particles expressing SARS-COV-2 Spike protein were generated as described by Crawford et al. (Jour. of Virol., Vol. 95. No. 24). In brief. 3×106 HEK-293T cells were co-transfected with a plasmid containing a lentiviral backbone expressing luciferase and ZsGreen (BEI catalog number NR-52516), a lentiviral helper plasmid expressing HIV Gag-Pol (BEI catalog number NR-52517), a lentiviral helper plasmid expressing HIV Tat (BEI catalog number NR-52518), and a lentiviral helper plasmid expressing HIV Rev (BEI catalog number NR-52519) along with a plasmid expressing the Spike protein of SARS-COV-2 using jetPRIME transfection reagent (Polyplus-transfection: NY) per the manufacturer's instructions. The culture supernatant containing pseudovirus particles was harvested at 48 h post-transfection, by centrifugation at 1200 rpm for 10 min and filtration through a 0.45 μM filter to remove cellular debris and then stored at −80° C., freezer in aliquots for downstream applications. The viral titers were determined using engineered HEK-293T cells expressing the human ACE2 receptor. For this purpose. 12.500 HEK293T-hACE2 cells were seeded per well in a poly−1-lysine-coated 96-well plate. 24 h after seeding, lentiviral particles were serially diluted with complete DMEM supplemented with Polybrene (5 mg/ml), and 50 μl of each dilution were added in four replicate wells. 48 h post addition, pseudoviral transduction efficiency was determined by measuring firefly luciferase activity in cell lysates using a bright-glo luciferase assay system (Promega. Madison. WI: catalog number E2610). The luminescence was measured using a SpectraMax i3× multi-mode plate reader (Molecular Devices. San Jose. CA) and relative luminescence units (RLUs) were plotted against virus dilutions.

### Example 6—SARS-COV-2 Entry Inhibitor Screening Assay

For screening SARS-COV-2 entry inhibitors. 24 h before starting the assay. 20,000 HEK-293T-hACE2 cells were seeded per well in a poly−1-lysine-coated 96-well plate. On the day of the assay setup, different concentrations of the compounds were mixed with the lentiviral particles expressing SARS-COV-2 Spike protein (1.00×105 RLU/well) and incubated at 37° C. for 30 min followed by the addition of 50 μl lentiviral particle-compound complex in the cells supplemented with Polybrene (5 mg/ml). After 48 h, the activity of firefly luciferase was measured as described above to calculate the ability of the compounds to block the entry of transduced pseudoviral particles in HEK-293T-hACE2 cells.

### Example 7—Production and Titration of SARS-COV-2 Stocks

SARS-COV-2 isolates USA-WII/2020 (BEI catalog number NR-52384), hCoV-19/South Africa/KRISP-EC-K005321/2020 (BEI catalog number NR-54008), and hCoV-19/Scotland/CVR2224/2020) (BEI catalog number NR-53945) were passaged in Vero-STAT-1 knockout cells. The viral titer was determined using the plaque assay. In brief. Vero E6 cells were seeded in 6-well plates. After 24 h, cells were washed with sterile 1× PBS. The viral stock was serially diluted and added to the cells in duplicate with fresh media, and the plates were incubated at 37ºC for 1 h with occasional shaking every 15 min. Then. 2 ml of 0.5% agarose in minimal essential media (MEM) containing 5% FBS and antibiotics was added per well. Plates were incubated at 37° C., for 72 h. Then, the cells were fixed with 4% paraformaldehyde overnight, followed by removing the overlay and staining with 0.2% crystal violet to visualize PFU. All assays were performed in a BSL-3 laboratory setting. The viral stocks used for all antiviral assays were generated in passage 1-2 of the initial stock obtained from BEI.

### Example 8—Assessment of Antiviral Activity of Selected Compounds

The entry inhibitor compounds were screened for antiviral activity through various means. In the preferred embodiment. UNCN1T or Vero-STAT1 knockout cells were seeded in 96-well plates 24 h before infection at 20,000 cells/well, or 48 h prior to infection for Calu-3 cells at the same seeding density as before. Different compounds (see FIGS. 2A-2M) ranging between 0.001 UM and 10 μM were added to the cells 2 h prior to the infection. The cells were infected with 0.1 MOI of SARS-COV-2 using Opti-MEM I reduced serum medium (Thermo Fisher catalog number 31985062) and incubated for 1 h at 37° ° C., with 5% CO₂. For vehicle control, cells were treated with the same concentration of DMSO. Mock-infected cells received only Opti-MEM I reduced serum medium. At the end of the incubation of the virus, the inoculum was removed, cells were washed with 1×PBS three times, and fresh media was added supplemented with the same concentration of compounds. Culture supernatant was collected at 24 hpi (hour postinfection) and 48 hpi. The SARS-COV-2 viral load was quantified in the culture supernatant using RT-QPCR with primer probes targeting the E gene of SARS-COV-2 using PrimeDirect Probe RT-qPCR Mix (TaKaRa Bio USA. Inc) and Applied Biosystems QuantStudio3 real-time PCR system (Applied Biosystems. Waltham. MA. USA) per manufacturer's instructions. Primers and probes used for SARS-COV-2 RNA quantification were as follows: E_Sarbeco_F1: 5′-ACAGGTACGTTAATAGTTAATAGCGT-3′ (400 nM) (SEQ ID NO: 1). E_Sarbeco_R2: 5′-ATATTGCAGCAGTACGCACACA-3′ (400 nM) (SEQ ID NO: 2) and E_Sarbeco_P1: 5′-FAM-ACACTAGCCATCCTTACTGCGCTTCG-BHQ1-3′ (200 nM) (SEQ ID NO: 3) as recommended by the WHO. The SARS-COV-2 genome equivalent copies were calculated using quantitative PCR (qPCR) control RNA from heat-inactivated SARS-COV-2, isolate USA-WA1/2020 (BEI catalog number NR-52347). The percentage inhibition of SARS-COV-2 replication in formula (Ia) and formula (IIa) treated cells was calculated with respect to viral loads in vehicle control wells that received DMSO (considered 0% inhibition) and negative control wells (uninfected cells). IC₅₀ values were calculated using four-parameter variable slope sigmoidal dose-response models using Graph Pad Prism 8.0 software.

### Example 8—Time of Compound Addition Assay

Vero-STAT1 knockout cells were seeded in 24-well plates and incubated overnight. The next day, formula (Ia) (5 μM) and formula (IIa) (5 μM) were added to the cells −2 h prior to infection, during infection (0) h), and +4 hpi. Then cells were infected with 0.1 MOI SARS-COV-2. The culture supernatants were collected at 24 hpi, and percent inhibition of viral replication was calculated under different exposure conditions using RT-qPCR.

### Example 9—Measuring the Combinational Antiviral Potential of Formula (Ia)/Formula (IIa) and RDV

To determine the possible synergistic antiviral effect of formula (Ia)/RDV and formula (IIa)/RDV against SARS-COV-2 replication, we tested combined doses of formula (Ia)/RDV and formula (IIa)/RDV in SARS-COV-2-infected UNCN1T cells. For these assays, the cells were seeded in 96-well plates (20,000 cells/well) 24 h before infection and then treated with respective combinations of compounds 2 h before infection. The cells were infected with 0.1 MOI of SARS-COV-2 isolate USA-WII/2020 as described above. The percentage inhibition of viral replication for each dose-combination was determined by RT-qPCR as described above. The percent inhibition of viral replication for a 1:1 fixed-dose combination of the compounds was used to generate dose-response plots. The CI was calculated using multiple drug effect equations developed by Chou and Talalay using the CompuSyn algorithm (https://www.combosyn.com). CI values of <1 indicate synergy. CI values >1 indicate antagonism, and values equal to 1 indicate additive effects (30, 31). Dose-response percent inhibition matrix of single and combined treatment of RDV/formula (Ia) and RDV/formula (IIa) in SARS-COV-2 infected UNCN1T Vero-STAT1 knockout cells 24 hpi and 3-D interaction landscape were calculated based on Loewe additive model using Synergy Finder v.2 (32).

### Example 10—Microscale Thermophoresis (MST) Assay

A one-to-one ratio of S-RBD and ACE2 was used in the microscale thermophoresis (MST) assays to determine the binding affinity of the compounds with S-RBD/ACE2 complex. The same MST method was used to determine the binding affinity of the compounds with S-RBD alone (FIG. 11A) and ACE2 alone (FIG. 11B). To prepare S-RBD for the assay, the receptor binding domain of the Spike protein (S-RBD) representing ancestral Wuhan-Hu-1 containing 6xHis-N-terminal tag was cloned, expressed, and purified to near homogeneity. Where needed, the 6xHis-tag was cleaved by TEV protease at the site inserted between the S-RBD and 6xHis-tag. The ACE2 was purchased from commercial vendors (Abcam and/or Acrobiosystems). A one-to-one ratio of S-RBD and ACE2 was used in the microscale thermophoresis (MST) assays to determine the binding affinity of the compounds with S-RBD/ACE2 complex. To monitor fluorescence change with temperature change (MST assay), either S-RBD or ACE2 was labelled with Monolith NT™ His-Tag Labeling Kit RED-tris-NTA MO-L008 (NanoTemper). The data were fit to a quadratic equation to obtain K_{d} (binding affinity). This same approach was used to measure the binding affinity of formula (IIa) binding with Hu-1 (FIG. 12A) providing a K_{d} of 299 nM, formula (IIa) binding with Delta (FIG. 12B) providing a K_{d} of 200 nM, formula (IIb) binding with Hu-1 (FIG. 12C) providing a K_{d} of 31 nM, and formula (IIb) binding with Delta (FIG. 12D) providing a K_{d} of 90 nM.

### Example 11—Statistical Analysis

The CC₅₀ and IC₅₀ values were computed using four-parameter variable slope sigmoidal dose-response models using GraphPad Prism (version 8.0). The CI was calculated using Chou and Talalay's multiple drug effect equation using the CompuSyn algorithm (https://www.combosyn.com). The 3-D interaction landscape between RDV and formula (IIa) was calculated based on Loewe's additive model incorporated within Synergy Finder v.2.

### Example 12—Assessment of Inhibitory Activity of Selected Compounds

In another approach. Vero-STAT1 knockout cells (ATCC R; CCL-81-VHG™) were cultured in 10 mM HEPES buffer supplemented with DMEM containing 10% fetal bovine serum. 2 mM L-glutamine. (100 units/ml) penicillin, and (100 units/ml) streptomycin. The UNCN1T cells (a human bronchial epithelial cell line: Kerafast: cat #ENC011) were cultured in BEGM (Bronchial Epithelial Cell Growth) media (Lonza, cat #CC-3170) in FNC (Athena Enzyme Systems: cat #0407) coated plates. The cells were incubated at 37° C., with 5% CO₂. 20-30 hours before infection 10,000-30.000 cells/well were seeded in 96 well plates. Different concentration of formula (IIa) and formula (Ia) or formula (IIb) (10 μM. 5 μM. 1 μM. 0.5 μM. 0.1 μM. 0.01 μM and 0.001 μM) was added to the cells 2 hours before infection. The cell were infected with SARS-COV-2 (Isolate USA-WII/2020; BEI cat #NR-52384) at a 0.1 MOI of using Opti-MEM® I reduced serum medium (Thermo Fisher. Cat #31985062) followed by incubation 37° C., for 1 hour in 5% CO₂. For positive control, cells were treated with same amount of DMSO equivalent to the added drugs. Mock infected cells received only Opti-MEM® I reduced serum medium. At the end of incubation virus inoculum was removed, cells were washed with 1×PBS for 3 times and fresh media was added supplemented with same concentration of drugs. Culture supernatant was collected at 24 hrs and 48 hrs post infection and SARS-COV-2 and the viral load was quantified using RT-QPCR with primer probes targeting E gene of SARS-COV-2 as described earlier (1). The SARS-COV-2 genome equivalent copies were calculated using quantitative PCR (qPCR) control RNA from the same heat-inactivated SARS-COV-2 isolate. The percent inhibition of SARS-COV-2 replication by formula (IIa), formula (Ia), or formula (IIb) compounds was calculated with respect to viral concentration in positive control wells that were treated with only DMSO (considered 0% inhibition) and negative control wells (uninfected cells). The cell IC₅₀ values were calculated using four parameter variable slope sigmoidal dose-response models using Graph Pad Prism 8.0 software.

### Example 13—Assessment of Cytopathic Effect

In some embodiments, cytopathic effect (CPE) was determined using the CellTiter-Glo luminescent cell viability assay (Promega: Madison, WI: cat #G9243) as per manufacturer's instructions (In this assay, the number of viable cells in culture was determined by quantifying ATP, which indicates the presence of metabolically active cells. Luminesence readout is directly proportional to the number of viable cells in culture). RLU values were plotted against log drug concentrations after normalization with RLU values from blank wells having no cells. Antiviral activity was determined by the degree of inhibition of viral replication. All experiments were carried out in triplicates, and repeated twice for by two different research personals.

It is to be understood that while the invention has been described in conjunction with the detailed description thereof, the foregoing description is intended to illustrate, and not limit the scope of the invention, which is defined by the scope of the appended claims. Other embodiments, advantages, and modifications are within the scope of the following claims. Any reference to accompanying drawings which form a part hereof, are shown, by way of illustration only. It is understood that other embodiments may be utilized and structural changes may be made without departing from the scope of the present disclosure. All publications discussed and/or referenced herein are incorporated herein in their entirety.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

1. A compound having one of the following formulae: 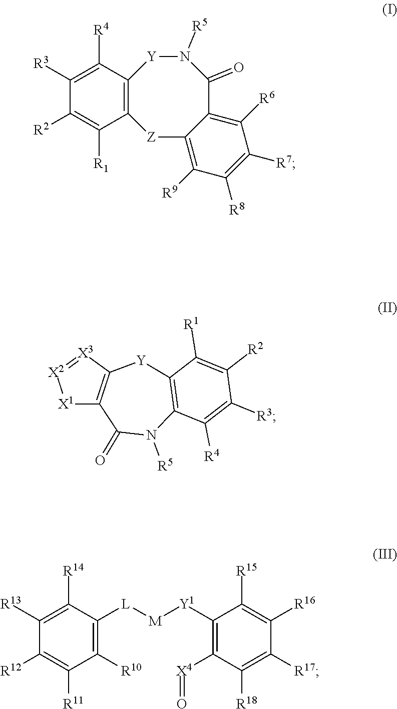
or
a pharmaceutically acceptable salt thereof for use in a medical therapy or a prophylactic treatment of a viral infection,
wherein each of R¹, R², R³, R⁴, R⁶, R⁷, R³ and R′ is independently hydrogen, halogen, nitro (—NO₂), aldehyde, carbonyl, carboxyl, hydroxyl, amine, aryl, heteroaryl, aryloxy, heteroaryloxy, —O(C₁-C₄)alkyl, —O(C₁-C₄)haloalkyl, (C₁-C₆)alkyl, or (C₁-C₆)alkyl substituted with one or more halogen;
R⁵ is independently hydrogen, halogen, aldehyde, carbonyl, carboxyl, hydroxyl, amine, aryl, heteroaryl, aryloxy, heteroaryloxy, (C₁-C₄)alkyl, (C₁-C₄)haloalkyl, —O(C₁-C₄)alkyl, or —O(C₁-C₄)haloalkyl;
each of R¹⁰, R¹¹, R¹², R¹³, R¹⁴, R¹⁵, R¹⁶, R¹⁷ and R¹⁸ is independently hydrogen, halogen, nitro (—NO₂), aldehyde, carbonyl, carboxyl, hydroxyl, amine, aryl, heteroaryl, aryloxy, heteroaryloxy, —O(C₁-C₄)alkyl, —O(C₁-C₄)haloalkyl, (C₁-C₆)alkyl, or (C₁-C₆)alkyl substituted with one or more halogen,
Y is O, S, S(═O), S(═O)₂, carbonyl, carboxyl, (C₁-C₆)alkyl, or (C₁-C₆)alkyl substituted with one or more halogen;
Z is O, S, S(═O), S(═O)₂, carbonyl, carboxyl, (C₁-C₆)alkyl, or (C₁-C₆)alkyl substituted with one or more halogen;
Y¹ is O, S, S(═O), S(═O)₂, nitro (—NO₂), aliphatic nitrile, carbonyl, carboxyl, (C₁-C₆)alkyl, or (C₁-C₆)alkyl substituted with one or more halogen;
X¹ is S, O, NH, or CR^{a1}, X² is N or CR^{a2}, X³ is N or CR^{a3}, and X⁴ is a (C₁-C₄)alkyl, wherein each of R^{a1}, R^{a2}, and R^{a3} is independently hydrogen, halogen, hydroxyl, (C₁-C₄)alkyl, (C₁-C₄)haloalkyl, aryl, heteroaryl, aryloxy, heteroaryloxy, —O(C₁-C₄)alkyl, or —O(C₁-C₄)haloalkyl;
L is absent or CR^{a4}, wherein R^{a4} is hydrogen, halogen, hydroxyl, (C₁-C₄)alkyl, (C₁-C₄)haloalkyl, aryl, heteroaryl, aryloxy, heteroaryloxy, —O(C₁-C₄)alkyl, or —O(C₁-C₄)haloalkyl; and
M is absent, NH, or N, wherein when M is N, M and X⁴ bind to form a cyclic group.

2. The compound of claim 1, wherein each of R¹, R², R³, R⁴, R⁶, R⁷, R⁸ and R⁹ is independently hydrogen, nitro (—NO₂), O(C₁-C₄)alkyl, —O(C₁-C₄)haloalkyl, (C₁-C₆)alkyl, or (C₁-C₆)alkyl substituted with one or more halogen;
R⁵ is independently hydrogen, halogen, (C₁-C₄)alkyl, (C₁-C₄)haloalkyl, —O(C₁-C₄)alkyl, or —O(C₁-C₄)haloalkyl;
each of R¹⁰, R¹¹, R¹², R¹³, R¹⁴, R¹⁵, R¹⁶, R¹⁷ and R¹⁸ is independently hydrogen, halogen, nitro (—NO₂), NH₂, O(C₁-C₄)alkyl, —O(C₁-C₄)haloalkyl, (C₁-C₆)alkyl, or (C₁-C₆)alkyl substituted with one or more halogen,
Y is O, S, S(═O), or S(═O)₂;
Z is O, S, S(═O), or S(═O)₂;
Y1 is O, S, S(═O), S(═O)₂, nitro (—NO₂), or aliphatic nitrile;
X¹ is S, O, or CR^{a1}, X² is N or CR^{a2}, X³ is N or CR^{a3}, and X⁴ is a (C₁-C₄)alkyl, wherein each of R^{a1}, R^{a2}, and R^{a3} is independently hydrogen, halogen, (C₁-C₄)alkyl, (C₁-C₄)haloalkyl, —O(C₁-C₄)alkyl, or —O(C₁-C₄)haloalkyl;
L is absent or CR^{a4}, wherein R^{a4} is hydrogen, (C₁-C₄)alkyl, (C₁-C₄)haloalkyl, —O(C₁-C₄)alkyl, or —O(C₁-C₄)haloalkyl; and
M is absent, NH, or N, wherein when M is N, M and X⁴ bind to form a cyclic group.

3. (canceled)

4. The compound of claim 1, wherein the compound of formula (I) is

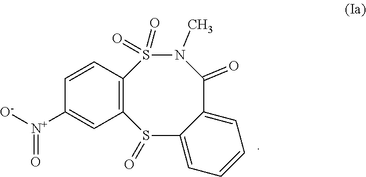 [SMILES: CN1C(=O)c2ccccc2S(=O)c2cc([N+](=O)[O-])ccc2S1(=O)=O]

5. The compound of claim 1, wherein the compound of formula (II) is

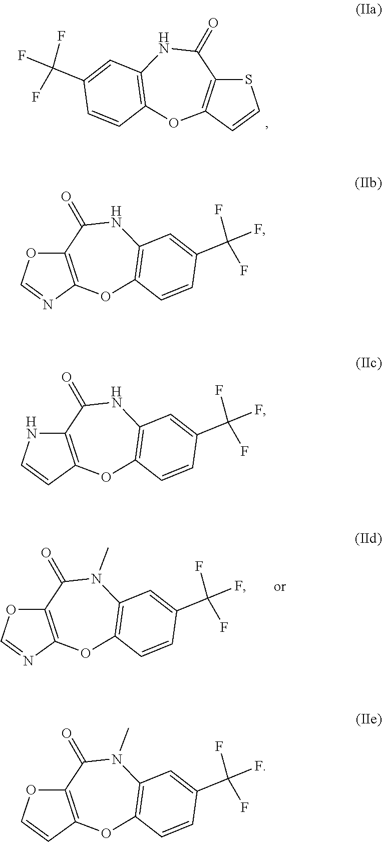

6. The compound of claim 1, wherein the compound of formula (III) is

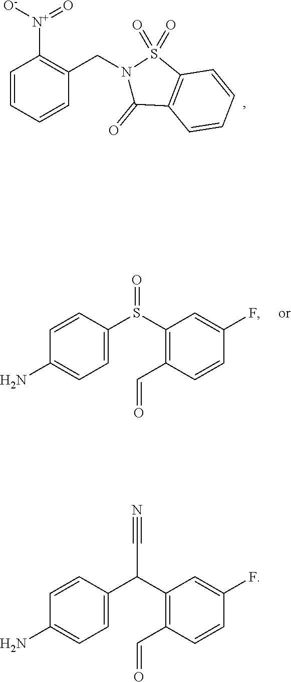

7. A pharmaceutical composition, comprising:
the compound of claim 1; and
a pharmaceutically acceptable carrier.

8. A method for preventing and/or treating a viral infection in a mammal in need thereof, comprising:
administering to a mammal an effective amount of the pharmaceutical compound of claim 1, wherein the pharmaceutical compound or pharmaceutically acceptable salt thereof binds to an interface of a SARS-COV-1 or SARS-COV-2 spike protein receptor binding domain (RBD) and a host cell ACE-2 receptor.

9. The method of claim 8, wherein the viral infection comprises infection with SARS-COV-1, SARS-COV-2, MERS-COV, NL63-COV, 229E-COV, OC43-COV, HKU1-COV, WIV1-COV, MHV, HKU9-COV, PEDV-COV, and/or SDCV.

10. The method of claim 9, wherein the mammal is human.

11. The method of claim 10, wherein the compound or pharmaceutical composition is administered orally, intraperitoneally, or intravenously.

12. The method of claim 11, wherein the compound or pharmaceutical composition is administered by a non-oral route.

13. The method of claim 12, wherein a therapeutically effective amount is an amount that blocks the replication of a SARS-COV-1 or SARS-COV-2 virus.

14. The method of claim 13, wherein a therapeutically effective amount is an amount that prevents entry of a SARS-COV-1 or SARS-COV-2 virus mediated by a spike protein of the SARS-COV-1 or SARS-COV-2 virus into a cell of the mammal.

15. The method of claim 14, wherein the compound of formula (II) is administered and comprises an entry inhibitor for the SARS-COV-1 or SARS-COV-2 virus.

16. The method of claim 15, wherein an additional antiviral agent is administered in combination with the compound of formula I, II, or III.

17. The method of claim 16, wherein the additional antiviral agent is a nucleoside analogue.

18. The method of claim 17, wherein the nucleoside analogue is remdesivir or ribavirin.

19. The method of claim 18, wherein the compound or pharmaceutical composition inhibits viral replication and/or prevents entry of the SARS-COV-1 or SARS-COV-2 virus mediated by the spike protein of the SARS-COV-1 or SARS-COV-2 virus into the cell at a nanomolar range.

20. (canceled)

21. A method of treating a SARS-COV-2 virus, comprising administering a compound to the subject, wherein the compound binds to SARS-COV-2 S-RBD/ACE2 Complex but does not bind to S-RBD alone or ACE-2 alone, wherein the compound comprises formula (IIa), or a pharmaceutically acceptable salt thereof.

22. The method of claim 2I, wherein the compound of formula (II) is [chemical structure]


SEQUENCE LISTING (3 sequences)

SEQ ID NO: 1 - DNA, 26 bp, Artificial Sequence
  synthetic construct
acaggtacgttaatagttaatagcgt

SEQ ID NO: 2 - DNA, 22 bp, Artificial Sequence
  synthetic construct
atattgcagcagtacgcacaca

SEQ ID NO: 3 - DNA, 26 bp, Artificial Sequence
  synthetic construct
acactagccatccttactgcgcttcg